(19) United States
(12) Patent Application Publication
(10) Pub. No.: US 2013/0330328 A1
(43) Pub. Date: Dec. 12, 2013

(54) Combination Therapy For B Cell Lymphomas

(71) Applicants: Ronald Herbst, Gaithersburg, MD (US); Elizabeth K. Ward, Gaithersburg, MD (US); Kathleen Phillips McKeever, Gaithersburg, MD (US)

(72) Inventors: Ronald Herbst, Gaithersburg, MD (US); Elizabeth K. Ward, Gaithersburg, MD (US); Kathleen Phillips McKeever, Gaithersburg, MD (US)

(73) Assignee: Medimmune, LLC, Gaithersburg, MD (US)

(21) Appl. No.: 13/885,219

(22) PCT Filed: Nov. 14, 2011
(86) PCT No.: PCT/US11/60520

§ 371 Date: Aug. 19, 2013

Related U.S. Application Data
(60) Provisional application No. 61/413,771, filed on Nov. 15, 2010.

(51) Int. Cl. A61K 39/395

(52) U.S. Cl. CPC A61K 39/39558
USPC 424/133.1; 424/173.1

(57) ABSTRACT

The present disclosure provides methods for treating B cell lymphomas using a combination of anti-CD19 and anti-CD20 antibodies. Such methods provide therapeutic advantages over single antibody therapies, including prolonged anti-tumor activity and/or reduced dosages.

### BACKGROUND

The majority of human leukemias and lymphomas, including acute lymphoblastic leukemia (ALL), chronic lymphocytic leukemia (CLL) and non-Hodgkin lymphoma (NHL) are of B-cell origin. In recent years, therapeutic approaches based on B cell depletion by targeting B cell restricted surface antigens with monoclonal antibodies (mAbs) have gained increasing attention. In particular, the anti-CD20 mAb rituximab has shown promising results in the treatment of B cell malignancies. (Robak et al., Cancer Treatment Reviews, 2007; 33:710-728.) The activity of rituximab largely depends on the ability of the mAb to mediate antibody-dependent cellular cytotoxicity (ADCC) by engaging activating Fcγ receptors on the surface of effector cells, such as macrophages and NK cells. (Desjarlais et al., Drug Discovery Today, 2007; 12:898-910.) While other mechanisms, such as complement-dependent cytotoxicity (CDC) and direct induction of apoptosis may also play a role, ADCC appears to be the dominant mechanism by which rituximab mediates elimination of malignant as well as normal B cells in vivo. (Edwards et al., Nat Rev Immunol, 2006; 6:394-403.) The combination of rituximab with chemotherapy has led to significant improvements in patients with follicular lymphoma (FL) and CLL and can result in long lasting remissions. However, this therapy is not effective across all patient populations. Thus, there still remains a great need for new and improved treatment options.

Human cluster of differentiation (CD) antigen 19 is a B cell-specific surface antigen that is expressed by early pre-B cells from the time of heavy chain rearrangement. CD19 belongs to the immunoglobulin domain-containing superfamily of transmembrane receptors. CD19 is expressed on B cells throughout their lineage from pro-B cells up to the plasma cell stage, when CD19 expression is down regulated. (Nadler et al., J Immunol. 1983; 131:244-250.) As a B cell-specific protein, and component of the B cell-receptor (BCR) complex, CD19 is a positive regulator of B cell-signaling that modulates the threshold for B cell activation and humoral immunity. (Sato et al., Proc Natl Acad Sci, 1995; 92:11558-62; Sato et al., J Immunol. 1997; 158:4662-9.) CD19 is not expressed on hematopoietic stem cells or on B cells before the pro-B-cell stage. (Nadler et al., J Immunol. 1983; 131:244-250; Loken et al., Blood, 1987; 70:1316-1324).

Importantly, expression of CD19 is maintained following malignant transformation of B cells, and CD19 is expressed on the majority of B cell malignancies, including ALL, CLL, and NHL. (Uckun et al., Blood, 1988; 71:13-29; D'Arena et al., Am J of Hematology, 2000; 64:275-281; Ginaldi et al., J Clin Pathol. 1998; 51:364-369; Anderson et al., Blood, 1984; 63:1424-1433.) The widespread and relatively stable expression of CD19 on B-cell malignancies makes this antigen an attractive target for mAb based therapies.

Thus there is a need for a combination therapy that utilizes the advantages of both anti-CD19 and anti-CD20 antibodies for treatment of B-cell malignancies.

### SUMMARY OF INVENTION

Provided herein are methods for treating B cell lymphomas that involve administering to a patient in need thereof a combination therapy comprising an anti-CD19 antibody and an anti-CD20 antibody. Such combination therapies confer anti-tumor activity for a longer duration than either said anti-CD19 antibody or said anti-CD20 antibody administered singly on a comparable dosing schedule.

Also provided are methods for treating B cell lymphomas utilizing a reduced dose of anti-CD19 and/or anti-CD20 antibodies. In particular, such methods involve administering to a patient in need thereof a combination therapy comprising an anti-CD19 antibody and an anti-CD20 antibody, wherein a dosage of said combination therapy has greater anti-tumor activity than a dosage of said anti-CD19 antibody that is at least two-fold higher than the dosage of the combination therapy.

### BRIEF DESCRIPTION OF THE DRAWINGS

FIG. 1 shows ADCC activity of the afucosylated anti-CD19 mAb 16C4 (16C4-afuc) and the anti-CD20 mAb, rituximab, against B cell leukemia and lymphoma cell lines. Results from in vitro ADCC assays with four cell lines are shown, which are representative for the activity profiles observed across a panel of 15 leukemia and lymphoma lines. The fucosylated CD19 mAb 16C4 was included for comparison. The ADCC activities of 16C4-afuc and rituximab were comparable when tested against Karpas-1106P cells (FIG. 1A); similar results were obtained with the cell lines Farage, Raji and MeC2. JVM2 (FIG. 1B) is representative of cell lines against which rituximab was more effective than 16C4-afuc in vitro: similar results were obtained with the cell lines Granta-519, DB, and JVM-13. Oci-LY19 (FIG. 1C) and Daudi (FIG. 1D) are representative for cell lines against which 16C4-afuc was more potent than rituximab; similar results were obtained with Toledo. Karpas-422, Nalm-6, RL, and Namalwa cells. With all cell lines tested 16C4-afuc was more potent than the fucosylated version of 16C4. The results shown are mean+/− standard deviation of triplicate samples. FIG. 1F-H shows relative expression of CD19 and CD20 (expressed as MFI; see Table I in Example 2) plotted against the % maximal cell kill observed (CD19, FIG. 1F; CD20, FIG. 1G) and against the EC50 values (CD9, FIG. 1H; CD20, FIG. 1I) determined for 16C4-afuc and rituximab. Cell line/mAb combinations for which an EC50 could not be determined were excluded (see also Table I in Example 2). Across this diverse panel of cell lines no significant correlation of EC50 or maximal cell killing with antigen expression could be determined.

FIG. 2 illustrates how patient-derived chronic lymphocytic leukemia (CLL) cells are sensitive to 16C4-afuc mediated ADCC in vitro. In FIG. 2A, Patient CLL cells show variable cell surface expression of CD19 and CD20. The number of CD19 and CD20 antigenic sites on CLL cells was determined as described below under Materials and Methods. In FIG. 2B-C, results from in vitro ADCC assays of 16C4-afuc and rituximab with patient-derived CLL cells using a FACS-based assay are shown. KC1333 NK cells were used as effector cells at an E:T ratio of 2.5:1. Shown are the results with three representative patient samples out of six samples tested in total. With all samples tested 16C4-afuc was more effective than rituximab in depleting CLL B cells from patient PBMC samples. All ADCC assays were performed in triplicate and mean values (+/− standard deviation) are presented. The relationship of antigen expression with in vitro ADCC activity for 16C4-afuc (FIG. 2E) and rituximab (FIG. 2F) for all six CLL patient samples tested is shown. The number of antigenic sites for CD19 and CD20 is plotted versus the maximum percent of cell kill achieved with 16C4 and rituximab, respectively.

FIG. 3 shows that patient-derived acute lymphoblastic leukemia (ALL) cells are sensitive to 16C4-afuc mediated ADCC. FIG. 3A illustrates expression of CD19 and CD20 on patient ALL cells. The number of antigenic sites for CD19 and CD20 was determined for three individual ALL samples. For comparison, the number of antigenic sites on normal human peripheral blood B cells from four individual donors is shown. As shown in FIGS. 3B, 3C, 3D, and 3E, 16C4-afuc has potent in vitro ADCC activity against primary ALL cells. The results from FACS-based assays with samples from four patients are shown. KC1333 NK cells were used as effector cells at an E:T ratio of 2.5:1; rituximab was included for comparison. All measurements were performed in triplicate with mean values (+/− standard deviation) presented.

FIG. 4 shows that tumor growth inhibition by 16C4-afuc is dependent on Fc-mediated effector function. In vivo tumor growth inhibition with 16C4-afuc and the effector-less Fc-mutated mAb 16C4-TM in Daudi (FIG. 4A) and Raji (FIG. 4B) SCID-lymphoma xenograft models are shown. SCID mice were inoculated s.c. with lymphoma cells on day 0. Beginning on day 7, animals received three weekly doses of mAb (2.5 mg/kg) or equal volume of vehicle (PBS). The human IgG1 mAb R347 was used as isotype control.

FIG. 5 shows that inhibition of lymphoma growth in SCID mice by 16C4-afuc is dose dependent. Treatment of Raji cell xenografts with a range of mAb concentrations and dosing frequencies caused significant tumor growth inhibition when compared with isotype control-treated animals. The dose range included 0.3, 1, 3, and 10 mg/kg 16C4-afuc. The dosing schedule variations included 1, 3, and 5 doses. The first dose was given on day 5 after cell implantation. In FIG. 5A, 5 doses of 16C4-afuc administered semiweekly resulted in stronger antitumor activity than 3 doses administered weekly (FIG. 5B). Treatment with 3 mg/kg achieved efficacy comparable to treatment with 10 mg/kg.

FIG. 6 shows the CD19 mAb 16C4-afuc is active in multiple SCID lymphoma models. FIG. 6A shows tumor growth inhibition with 16C4-afuc and rituximab in three s.c. lymphoma xenograft models. Shown are results for Namalwa (FIG. 6A), Daudi (FIG. 6B) and Toledo (FIG. 6C) B lymphoma cell lines. SCID mice were implanted s.c. with tumor cells on day 0 then injected twice weekly i.p. with 16C4-afuc, isotype control, vehicle, or rituximab beginning on day 5 for a total of 5 doses (3 mg/kg). In FIGS. 6D and 6E, the CD19 mAb 16C4-afuc is active in mouse models of disseminated disease. Comparison of the anti-tumor activity of 16C4-afuc and rituximab in two systemic disease models is shown. The results shown are for mice injected with Namalwa (FIG. 6A) and Daudi (FIG. 6B) cells. Twice weekly i.p. administration of 16C4-afuc, rituximab, or isotype control (3 mg/kg) started on day 7 post cell injection and continued for 5 doses. In the disseminated tumor model, survival time or the time to paralysis was used as endpoint.

FIG. 7 shows the effects of prolonged suppression of tumor growth in SCID lymphoma models by combination of 16C4-afuc and rituximab. In SCID-lymphoma models rituximab was administered according to the same schedule and concentration as 16C4-afuc (3 mg/kg semiweekly for a total of five doses). Results from Raji (FIG. 7A), Daudi (FIGS. 7B), Oci-LY19 (FIG. 7C) and Ramos (FIG. 7D) xenograft models are shown. The combination of 16C4-afuc with rituximab resulted in prolonged suppression of tumor growth for Raji and Daudi models. A lesser effect was observed in the Oci-LY19 model, which responded poorly to rituximab, and the Ramos model, which responded poorly to 16C4-afuc.

FIG. 8 shows the pharmacokinetics and pharmacodynamics of 16C4-afuc, administered either alone or in combination with rituximab in huCD19/CD20 transgenic mice. Four separate administrations were considered: 16C4-afuc (1 mg/kg)+control; 16C4-afuc (10 mg/kg)+control; 16C4-afuc (1 mg/kg)+rituximab; 16C4-afuc (10 mg/kg)+rituximab. The higher dose of 16C4-afuc (10 mg/kg), whether administered alone or in combination, was retained for longer in the blood than the lower dose of 16C4-afuc (1 mg/kg).

FIG. 9 shows the result of a B cell depletion experiment, following administration of either combination therapy, rituximab alone, or 16C4-afuc alone. The results indicate that the highest dose of combination therapy, with rituximab (10 mg/kg)+16C4-afuc (10 mg/kg), led to the greatest percentage of B cell depletion from the blood and spleen for the longest duration.

### DETAILED DESCRIPTION

### A. Combination Therapy with Anti-CD19 and Anti-CD20 Antibodies

Provided herein are methods for treating B cell malignancies that involve administering a combination of an anti-CD19 antibody and an anti-CD20 antibody. The combination therapy provides prolonged anti-tumor efficacy and/or effective treatment at a reduced dosage as compared to single antibody therapy (e.g., administration of either anti-CD19 or anti-CD20 alone). Also provided are methods for prolonging inhibition of tumor growth in a subject in need thereof (e.g., a subject suffering from a B cell malignancy) by administering to the subject a combination of an anti-CD19 antibody and an anti-CD20 antibody.

As used herein “treating” or “treatment” refers to the administration of an anti-CD19 antibody or antigen-binding fragment thereof to a subject, or administration of an anti-CD19 antibody or fragment thereof to an isolated tissue or cell line from a subject, in combination with the administration of an anti-CD20 antibody or antigen-binding fragment thereof to the subject, or to an isolated tissue or cell line from the subject, where the subject has a disease, a symptom of a disease, or a predisposition toward a disease, where the purpose is to cure, heal, alleviate, relieve, alter, remedy, ameliorate, improve, or affect the disease, the symptoms of the disease, or the predisposition toward the disease. By “treating” or “treatment” is also intended the combination of these antibodies or antigen-binding fragments thereof can be administered to the subject, or to the isolated tissue or cell line from the subject, as part of a single pharmaceutical composition, or alternatively as part of individual pharmaceutical compositions, each comprising either the anti-CD19 antibody (or antigen binding fragment thereof) or anti-CD20 antibody (or antigen-binding fragment thereof), where the subject has a disease, a symptom of a disease, or a predisposition toward a disease, where the purpose is to cure, heal, alleviate, relieve, alter, remedy, ameliorate, improve, or affect the disease, the symptoms of the disease, or the predisposition toward the disease.

Multiple parameters can be indicative of treatment efficacy, e.g anti-tumor activity. These include, but are not limited to, a reduction in the size of the tumor mass; a reduction in metastatic invasiveness of the tumor; a reduction in the rate of tumor growth; a decrease in severity or incidence of tumor-related sequelae such as cachexia and ascites production; a decrease and/or prevention of tumor-related complications such as pathologic bone fractures, autoimmune hemolytic anemia, prolymphocytic transformation, Richter's syndrome, and the like; sensitization of the tumor to chemotherapy and other treatments; an increased patient survival rate: an increase in observed clinical correlates of improved prognosis such as increased tumor infiltrating lymphocytes and decreased tumor vascularization; and the like. Thus, in some embodiments, administration of the combination of these two types of antibodies will result in an improvement of one or more of these parameters in a patient (e.g., subject) undergoing treatment. In other embodiments, the improvements in the patient will be synergistic with regard to some parameters, but additive with regard to others.

In certain embodiments, the effect of combination therapy with CD19 and CD20 antibodies may be additive. In other embodiments, the effects of combination therapy with CD19 and CD20 antibodies may be synergistic. The term “synergy” is used to describe a combined effect of two or more active agents that is greater than the sum of the individual effects of each respective active agent. Thus, where the combined effect of two or more agents results in “synergistic inhibition” of an activity or process, for example, tumor growth, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies. For the purposes herein, synergistic effect means that the effect observed when employing a combination of a CD19 antibody and a CD20 antibody administered on a comparable dosing schedule or regime is (1) greater than the effect achieved when that CD19 antibody or CD20 antibody is employed alone (or individually) and (2) greater than the sum added (additive) effect for that CD19 antibody and CD20 antibody. As used herein, comparable dosing schedule refers to a dosing schedule or regime that is used to evaluate or compare the results of at least two different treatments and as such is designed to be the same as between the treatments being compared, i.e. patients are being dosed in the same way (e.g., day, time between dosing, concentration of antibody agent) but with a different antibody or combination therapy. The variables of the dosing schedule will be determined by one of skill in the art depending on the B cell malignancy being treated and choice of treatment. Such synergy or synergistic effect can be determined by way of a variety of means known to those in the art. For example, the synergistic effect of a CD19 antibody and a CD20 antibody can be observed using in vitro or in vivo assay formats examining reduction of tumor cell number or tumor size, or by inhibition of tumor growth or a depletion of tumor cells. Similarly, a synergistically effective amount of each individual component may be determined by testing a range of concentrations of each component.

As used herein the term “combination” is used in its broadest sense and means that a subject is treated with at least two therapeutic regimens. Thus, “combination antibody therapy” or “combination therapy” is intended to mean a subject is treated with at least two antibody regimens, more particularly, with at least one anti-CD20 antibody (or antigen-binding fragment thereof) in combination with at least one anti-CD19 antibody (or antigen-binding fragment thereof), but the timing of administration of the different antibody regimens can be varied so long as the beneficial effects of the combination of these antibodies is achieved. Treatment with an anti-CD20 antibody (or antigen-binding fragment thereof) in combination with an anti-CD19 antibody (or antigen-binding fragment thereof) can be at the same time (e.g. simultaneously or concurrently), or at different times (e.g. consecutively or sequentially), or a combination thereof. For the purposes of the present disclosure, administering at the same time (e.g., simultaneously) refers to administering the antibodies together in same formulation or in separate formulations wherein the administration may be a few minutes to a few hours apart, but no more than one day. As used herein administering at different times (e.g., sequentially) refers to administering the antibodies of the combination therapy a few hours to days, weeks and even months apart.

Therefore, in certain embodiments a subject undergoing combination antibody therapy can receive both antibodies at the same time (e.g., simultaneously) or at different times (e.g., sequentially, in either order, on the same day, or on different days), so long as the therapeutic effect of the combination of both substances is caused in the subject undergoing therapy. In some embodiments, the combination of antibodies will be given simultaneously for one dosing, but other dosings will include sequential administration, in either order, on the same day, or on different days. Sequential administration may be performed regardless of whether the subject responds to the first monoclonal antibody administration. Where the two antibodies are administered simultaneously, they can be administered as separate pharmaceutical compositions, each comprising either the anti-CD20 antibody (or antigen-binding fragment thereof) or the anti-CD19 antibody (or antigen-binding fragment thereof), or can be administered as a single pharmaceutical composition comprising both of these antibodies.

The methods of the disclosure comprise using combination therapy which confers a positive therapeutic response to a subject in need thereof a treatment for B cell diseases. A positive therapeutic response with respect to the combination treatment using anti-CD19 and anti-CD20 antibodies is intended to mean an improvement in the disease in association with the anti-tumor activity of these antibodies or fragments thereof, and/or an improvement in the symptoms associated with the disease. That is, an anti-proliferative effect, the prevention of further tumor outgrowths, a reduction in tumor size, a reduction in the number of cancer cells, and/or a decrease in one or more symptoms mediated by neoplastic B cells can be observed. Thus, for example, an improvement in the disease may be characterized as a complete response. By “complete response” is intended an absence of clinically detectable disease with normalization of any previously abnormal radiographic studies, bone marrow, and cerebrospinal fluid (CSF). Such a response must persist for at least one month following treatment according to the methods of the disclosure. Alternatively, an improvement in the disease may be categorized as being a partial response. By “partial response” is intended at least about a 50% decrease in all measurable tumor burden (e.g. the number of tumor cells present in the subject) in the absence of new lesions and persisting for at least one month. Such a response is applicable to measurable tumors only.

Tumor response can be assessed for changes in tumor morphology (e.g., overall tumor burden, tumor size, and the like) using screening techniques such as magnetic resonance imaging (MRI) scan, x-radiographic imaging, computed tomographic (CT) scan, flow cytometry or fluorescence-activated cell sorter (FACS) analysis, bioluminescent imaging, for example, luciferase imaging, bone scan imaging, and tumor biopsy sampling including bond marrow aspiration (BMA). In addition to these positive therapeutic responses, the subject undergoing therapy may experience the beneficial effect of an improvement in the symptoms associated with the disease. Thus, for B cell tumors, the subject may experience a decrease in the so-called B symptoms, e.g., night sweats, fever, weight loss, and/or urticaria.

The combination therapy disclosed herein is administered at a therapeutically effective dose. The term “therapeutically effective dose,” “therapeutically effective amount,” or “effective amount” is intended to be an amount of the anti-CD19 antibody (or antigen-binding fragment thereof) that, when administered in combination with an amount of the anti-CD20 antibody (or antigen-binding fragment thereof), brings about a positive therapeutic response with respect to treatment of a subject for a cancer comprising neoplastic B cells. In some embodiments, a therapeutically effective dose of either the anti-CD20 antibody (or antigen-binding fragment thereof) or anti-CD19 antibody (or antigen-binding fragment thereof) is in the range from about 1 mg/kg to about 200 mg/kg. It is recognized that the method of treatment may comprise a single administration of a therapeutically effective dose of the antibody combination useful in the practice of the methods or multiple administrations of a therapeutically effective dose of the antibody combination.

In certain embodiments, the combination therapy with anti-CD19 antibody and an anti-CD20 antibody provides prolonged anti-tumor activity relative to treatments involving either an anti-CD19 antibody alone or an anti-CD20 antibody alone. In certain embodiments, a combination therapy may provide anti-tumor activity that lasts for at least 1 week, 2 weeks, 3 weeks, 1 month, 2 months, 3 months, 4 months, 5 months, 6 months, 1 year, or 2 years longer than the anti-tumor activity obtainable with either an anti-CD19 antibody alone or an anti-CD20 antibody alone. The relative duration of anti-tumor activity may be determined based on statistical analysis of a test population. For example, if an anti-CD19 antibody exhibits a certain level of anti-tumor activity for a mean of 6 weeks in a test population and an anti-CD20 antibody exhibits a certain level of anti-tumor activity for a mean of 8 weeks in a test population, then the combination exhibits anti-tumor activity that is at least 4 weeks longer than either antibody therapy administered alone if the combination therapy exhibits at least the same level of anti-tumor activity as that seen for the single antibody therapies for a mean of at least 12 weeks in a test population. The duration of anti-tumor activity may be measured as the date on which therapy begins until the time point at which the therapy is no longer providing a desired level of anti-tumor activity (e.g., as measured by the ability to prevent an increase in tumor volume, the ability to deplete B cells, etc.).

In certain embodiments, anti-tumor activity as described herein refers to the ability to prevent more than a 1%, 2%, 5%, 8%, 10%, 12%, 15%, 20%, 25% or 30% increase in tumor size. As used herein, tumor size refers to the diameter measurement or estimated tumor volume measurement. For example, if a combination therapy can prevent a tumor from increasing by more than 10% in uni- or bi-dimensional measurements over a six month period it exhibits anti-tumor activity over a six month period. These dimensional measurements of lymphoma tumors (e.g. tumors present in the peripheral lymph nods) can be obtained by body scans, using instruments described above, that provide diameter or estimated tumor volume measurements (Journal of Clinical Oncology, 2004 ASCO Annual Meeting Proceedings (Post-Meeting Edition). Vol 22, No 14S (July 15 Supplement), 2004: 6606)). Therefore, the presently disclosed combination therapy exhibits anti-tumor activity when there is a lack of an increase in either a diameter measurement of a tumor or in an estimated tumor volume measurement. In another embodiment, anti-tumor activity may be reflected in an actual decrease in tumor size, or maintenance of a tumor at a fixed size over a period of time. In another embodiment, anti-tumor activity may be determined by inhibition of tumor growth of more than a 1%, 2%, 5%, 8%, 10%, 12%, 15%, 20%, 25% or 30% relative to the size of tumor before treatment, e.g. diameter measurements or estimated tumor volume measurements.

In certain embodiments, anti-tumor activity may be measured by determining the level of B cell depletion obtained with a given therapy. Circulating B cells, including malignant B cells, are most easily measured by flow cytomery, or other cell counting devices described above and well known in the art, resulting in a count number of circulating B cells. It is further contemplated that any method providing for a number of circulating B cells can be used to determine the depletion of B cells after treatment with the combination therapy. Circulating B cell depletion is well understood to be a surrogate marker for tissue B cells depletion. Therefore, such B cell depletion includes both circulating B cells and tissue B cells, some of which may be malignant. In certain embodiments, anti-tumor activity may be determined by a level of B cell depletion of at least 50%, 60%, 70%, 75%, 80%, 85%, 90%, 95%, 97%, 98%, 99%, or 100% relative to the level of B cells before treatment. For example, if a therapy can maintain a level of B cell depletion of 90% over a period of at least six months, the therapy exhibits anti-tumor activity over a six month period.

One method of predicting clinical efficacy is to measure the effects of combination therapy with these antibodies in a suitable model; for example, the use of the combination of an anti-CD20 antibody and an anti-CD19 antibody in murine cancer models. These models include the nude mouse xenograft tumor models such as those using the human Burkitt's lymphoma cell lines known as Namalwa and Daudi. In some embodiments, anti-tumor activity is assayed in a staged nude mouse xenograft tumor model using the Daudi human lymphoma cell line. A staged nude mouse xenograft tumor model cell line is generally more effective at distinguishing the therapeutic efficacy of a given antibody than is an unstaged model, as in the staged model antibody dosing is initiated only after the tumor has reached a measurable size. In the unstaged model, antibody dosing is initiated generally within about 1 day of tumor inoculation and before a palpable tumor is present. The ability of an antibody to exhibit increased anti-tumor activity in a staged model is a strong indication that the antibody will be therapeutically effective.

In other embodiments, a combination of an anti-CD19 antibody and an anti-CD20 antibody provides methods for inhibiting tumor growth or treating patients having B cell malignancies using reduced dosages of the therapeutic antibodies. In particular, the examples provided herein show that the total dose of a combination therapy (e.g., a combination of an anti-CD19 antibody and an anti-CD20 antibody) is more effective than a dose of an anti-CD19 antibody that is greater than the total dose of the combination therapy. Accordingly, the methods disclosed herein provide for enhancing the efficacy of a single antibody therapy thereby permitting effective treatment at lower dosages and potentially avoiding undesirable side effects associated with higher dosages of the antibody therapy. In certain embodiments, a combination therapy provides the same or greater anti-tumor effect at a total dose that is at least 2-fold, 3-fold, 4-fold or 5-fold lower than the dose of either an anti-CD19 antibody or an anti-CD20 antibody that would be required to give the same anti-tumor activity (e.g., the same degree of anti-tumor activity in response to a single dose, or the same degree of anti-tumor activity over a defined time period using a comparable dosing schedule). Thus, for any given dosage of anti-CD19 antibody that confers an anti-tumor activity, the combination therapy will confer a greater anti-tumor activity at a lower concentration than the dosage of the anti-CD19 antibody. The lower concentration may be half of the concentration of the anti-CD19 antibody, or may be less than one half of the concentration of anti-CD19 antibody.

In certain embodiments, the combination therapy described herein may be achieved by various means of administration. For example, the anti-CD 9 antibody and the anti-CD20 antibody may be separately formulated and administered to the patient. Alternatively, the anti-CD19 and anti-CD20 antibody may be formulated together in a single formulation. When the anti-CD19 and anti-CD20 antibodies are in separate formulations, the antibodies may be administered on the same or different dosing schedules. For example, the two antibodies may be administered at the same time and at the same frequency (for example, both antibodies administered at the same time once per week), they may be administered at separate times but on the same frequency of administration (for example, both antibodies are administered once per week, but at different times), or they may be administered using schedules that differ in frequency (for example, one antibody is administered once per week and the other antibody is administered every other week), etc. In exemplary embodiments, the anti-CD19 and anti-CD20 antibodies are formulated together and administered on the same dosing schedule.

### B. Anti-CD19 Antibodies

The combination therapy described herein comprises anti-CD19 antibodies. The term “CD19” or “CD19 antigen” refers to an antigen of about 90 kDa identified, for example, by the HD237 or B4 antibody (Kiesel et al., Leukemia Research 11, 12:1119 (1987)). CD19 is found on cells throughout differentiation of B-lineage cells from the stem cell stage through terminal differentiation into plasma cells, including but not limited to, pre-B cells, B cells (including naive B cells, antigen-stimulated B cells, memory B cells, plasma cells, and B lymphocytes) and follicular dendritic cells. CD19 is also found on B cells in human fetal tissue. In preferred embodiments, the CD19 antigen targeted by the antibodies disclosed herein is the human CD19 antigen.

Any suitable anti-CD19 antibody may be used in accordance with the methods and compositions described herein. Suitable anti-CD19 antibodies include, for example, known anti-CD19 antibodies, commercially available anti-CD19 antibodies, or anti-CD19 antibodies developed using methods well known in the art.

As used herein, the terms “antibody” and “antibodies”, also known as immunoglobulins, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies disclosed herein), intrabodies, and epitope-binding fragments of any of the above. In particular, antibodies include immunoglobulin molecules and immunologically active fragments of immunoglobulin molecules, e.g., molecules that contain at least one antigen-binding site.

A CD19 antibody of the disclosure may be a monoclonal human, humanized or chimeric anti-CD19 antibody. Anti-CD19 antibodies used in compositions and methods of the disclosure can be naked antibodies, immunoconjugates or fusion proteins. In certain embodiments, an anti-CD19 antibody of the disclosure may mediate human antibody-dependent cellular cytotoxicity (ADCC), complement-dependent cell-mediated cytotoxicity (CDC), and/or apoptosis in an amount sufficient to deplete circulating B cells. In exemplary embodiments, an anti-CD19 antibody of the disclosure is an anti-CD19 antibody that has been engineered to have enhanced ADCC activity relative to the parent antibody. Methods for creating antibody variants having enhanced ADCC activity are described further below. In certain embodiments, an anti-CD19 antibody of the disclosure is an afucosylated antibody having enhanced ADCC activity.

In certain embodiments, an anti-CD19 antibody used in the compositions and methods of the disclosure may be a human, humanized or chimeric antibody having an IgG isotype, particularly an IgG1, IgG2, IgG3, or IgG4 human isotype or any IgG1, IgG2, IgG3, or IgG4 allele found in the human population. Antibodies of the human IgG class have functional characteristics such as a long half-life in serum and the ability to mediate various effector functions (Monoclonal Antibodies: Principles and Applications, Wiley-Liss, Inc., Chapter 1 (1995)). The human IgG class antibody is further classified into the following 4 subclasses: IgG1, IgG2, IgG3 and IgG4. A large number of studies have so far been conducted for ADCC and CDC as effector functions of the IgG class antibody, and it has been reported that among antibodies of the human IgG class, the IgG1 subclass has the highest ADCC activity and CDC activity in humans (Chemical Immunology, 65, 88 (1997)).

In certain embodiments, an anti-CD19 antibody of the disclosure is a known anti-CD19 antibody including, but not limited to, HD37 (IgG1, kappa) (DAKO North America, Inc., Carpinteria, Calif.), BU12 (Callard et al., J. Immunology, 148(10):2983-7 (1992)), 4G7 (IgG1) (Meeker et al., Hybridoma, 3(4):305-20 (1984 Winter)). J4.119 (Beckman Coulter, Krefeld, Germany), B43 (PharMingen, San Diego, Calif.), SJ25C1 (BD PharMingen, San Diego, Calif.), FMC63 (IgG2a) (Zola et al., Immunol. Cell. Biol. 69(PT6): 411-22 (1991); Nicholson et al., Mol. Immunol., 34:1157-1165 (1997); Pietersz et al., Cancer Immunol. Immunotherapy, 41:53-60 (1995)), 89B(B4) (IgG1) (Beckman Coulter, Miami, Fla.; Nadler et al., J. Immunol., 131:244-250 (1983)), and/or HD237 (IgG2b) (Fourth International Workshop on Human Leukocyte Differentiation Antigens, Vienna, Austria, 1989; and Pezzutto et al, J. Immunol., 138(9):2793-2799 (1987)). In other embodiments, an anti-CD19 antibody of the disclosure is any of the anti-CD19 antibodies described in U.S. Publication Nos. 2008/0138336 and 2009/0142349 and U.S. Pat. Nos. 7,462,352 and 7,109,304. In exemplary embodiments, an anti-CD19 antibody is the 16C4 antibody, or an antigen binding fragment thereof, as described in U.S. Publication No. 2008/0138336 and below.

In certain embodiments, an anti-CD19 antibody is an isotype switched variant of a known anti-CD19 antibody (e.g., to an IgG1 or IgG3 human isotype) such as those described above.

In certain embodiments, an anti-CD19 antibody of the disclosure may immunospecifically bind to human CD19 and may have a dissociation constant (K_{d}) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using a method known to one of skill in the art (e.g., a BIAcore assay, ELISA) (Biacore International AB, Uppsala, Sweden). In certain embodiments, an anti-CD19 antibody of the disclosure may immunospecifically bind to a human CD19 antigen and may have a dissociation constant (K_{d}) of between 25 to 3400 pM, 25 to 3000 pM, 25 to 2500 pM, 25 to 2000 pM, 25 to 1500 pM, 25 to 1000 pM, 25 to 750 pM, 25 to 500 pM, 25 to 250 pM, 25 to 100 pM, 25 to 75 pM, 25 to 50 pM as assessed using a method known to one of skill in the art (e.g., a BIAcore assay, ELISA). In certain embodiments, an anti-CD19 antibody of the disclosure may immunospecifically bind to human CD19 and may have a dissociation constant (K_{d}) of 500 pM, 100 pM, 75 pM or 50 pM as assessed using a method known to one of skill in the art (e.g., a BIAcore assay, ELISA).

In certain embodiments, anti-CD19 antibodies for use in compositions and methods of the disclosure may be able to reduce or deplete B cells in a human treated therewith. Depletion of B cells can be in circulating B cells, or in particular tissues such as, but not limited to, bone marrow, spleen, gut-associated lymphoid tissues, and/or lymph nodes. In one embodiment, anti-CD19 antibody of the disclosure may deplete circulating B cells, blood B cells, splenic B cells, marginal zone B cells, follicular B cells, peritoneal B cells, and/or bone marrow B cells. In a one embodiment, an anti-CD19 antibody of the disclosure may achieve depletion of progenitor B cells, early pro-B cells, late pro-B cells, large-pre-B cells, small pre-B cells, immature B cells, mature B cells, antigen stimulated B cells, and/or plasma cells. Such depletion may be achieved via various mechanisms such as antibody-dependent cell-mediated cytotoxicity (ADCC), and/or by blocking of CD19 interaction with its intended ligand, and/or complement dependent cytotoxicity (CDC), inhibition of B cell proliferation and/or induction of B cell death (e.g., via apoptosis). By “depletion” of B cells it is meant a reduction in circulating B cells and/or B cells in particular tissue(s) by at least about 25%, 40%, 50%, 65%, 75%, 80%, 85%, 90%, 95% or more. In particular embodiments, virtually all detectable B cells are depleted from the circulation and/or particular tissue(s).

In certain embodiment, B cell depletion by an anti-CD19 antibody of the disclosure may persist for at least 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In another embodiment, B cell depletion by an anti-CD19 antibody of the disclosure may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In a further embodiment, B cell depletion by an anti-CD19 antibody of the disclosure may persist for at least 1 month, at least 2 months, at least 3 months, at least 4 months, at least 5 months, at least 6 months, at least 7 months, at least 8 months, at least 9 months, at least 10 months, at least 11 months or at least 12 months.

B cell malignancies are characterized by the pathological expansion of specific B cell subsets, for example, precursor B cell acute lymphoblastic leukemia is characterized by an abnormal expansion of B cells corresponding to pro-B cell/Pre-B cell developmental stages. The malignant B cells maintain cell surface expression of normal B cell markers such as CD19. An anti-CD19 antibody may therefore deplete malignant B cells in a human subject. In a specific embodiment, an anti-CD19 antibody of the disclosure may achieve at least 20%, at least 30%, at least 40%, at least 50%, at least 60%, at least 70%, at least 80%, at least 90%, at least 95%, or at least 100% depletion of malignant B cells in a human subject.

### Engineering Effector Function

In some embodiments, anti-CD19 antibodies are modified with respect to effector function, so as to enhance the effectiveness of the antibody in treating B cell malignancies, for example. An exemplary effector function is antibody-dependent cell-mediated cytotoxicity, or ADCC, which is a cell-mediated reaction in which non-specific cytotoxic cells recognize bound antibody on a target cell and subsequently cause lysis of the target cell. The cytotoxic cells, or effector cells, may be leukocytes which express one or more FcRs. Effector cells express at least FcγRI, FCγRII, FcγRIII and/or FcγRIV in mouse. Some human leukocytes which mediate ADCC include peripheral blood mononuclear cells (PBMC), natural killer (NK) cells, monocytes, cytotoxic T cells and neutrophils. Of these cells, the primary cells for mediating ADCC are NK cells, which express FcγRIII. Monocytes express FcγRI, FcγRII, FcγRIII and/or FcγRIV. FcR expression on hematopoietic cells is summarized in Ravetch and Kinet, Annu. Rev. Immunol., 9:457-92 (1991).

One method for enhancing effector function of antibodies is by producing engineered glycoforms. Engineered glycoforms may be generated by any method known to one skilled in the art, for example by using engineered or variant expression strains, by co-expression with one or more enzymes, for example DI N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180: Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684: U.S. Ser. No. 10/277,370; U.S. Ser. No. 10/113,929; PCT WO 00/61739A1; PCT WO 01/292246A1; PCT WO 02/311140A1; PCT WO 02/30954A 1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614: Okazaki et al., 2004, JMB, 336: 1239-49. One or more amino acid substitutions can also be made that result in elimination of a glycosylation site present in the Fc region (e.g., Asparagine 297 of IgG). Furthermore, aglycosylated antibodies may be produced in bacterial cells which lack the necessary glycosylation machinery.

An antibody can also be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. See, for example, Shields, R. L. et al. (2002) J. Biol. Chem. 277:26733-26740; Umana et al. (1999) Nat. Biotech. 17:176-1, as well as, U.S. Pat. No. 6,946,292; European Patent No: EP 1,176,195; PCT Publications WO 03/035835; WO 99/54342 each of which is incorporated herein by reference in its entirety.

In one embodiment, an anti-CD19 antibody of the disclosure comprises a variant Fc region that mediates enhanced antibody-dependent cellular cytotoxicity (ADCC). In one embodiment, an anti-CD19 antibody of the disclosure comprises an Fc region having complex N-glycoside-linked sugar chains linked to Asn297 in which fucose is not bound to N-acetylglucosamine in the reducing end, wherein said Fc region mediates enhanced antibody-dependent cellular cytotoxicity (ADCC).

In certain embodiments, an anti-CD19 antibody of the disclosure comprises an Fc variant, wherein said variant Fc domain has an affinity for Fc gamma receptor IIB that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable non-variant Fc domain.

In other embodiments, effector function may be altered by introducing cysteine residue(s) in the Fc region of the antibody, thereby allowing interchain disulfide bond formation in this region. The homodimeric antibody thus generated may have improved internalization capability and/or increased complement-mediated cell killing and/or antibody-dependent cellular cytotoxicity (ADCC). See, Caron et al., J. Exp Med., 176:1191-1195 (1992) and Shopes, B., J. Immunol., 148:2918-2922 (1992). Homodimeric antibodies with enhanced anti-tumor activity may also be prepared using heterobifunctional cross-linkers as described in Wolff et al., Cancer Research, 53:2560-2565 (1993). An antibody can also be engineered which has dual Fc regions and may thereby have enhanced complement lysis and ADCC capabilities. See, Stevenson et al., Anti-Cancer Drug Design, 3:219-230 (1989).

Other methods of engineering Fc regions of antibodies so as to alter effector functions are known in the art (e.g., U.S. Patent Publication No. 20040185045 and PCT Publication No. WO 2004/016750, both to Koenig et al., which describe altering the Fc region to enhance the binding affinity for FcγRIIB as compared with the binding affinity for FCγRIIA; see, also, PCT Publication Nos. WO 99/58572 to Armour et al., WO 99/51642 to Idusogie et al., and U.S. Pat. No. 6,395,272 to Deo et al.; the disclosures of which are incorporated herein in their entireties). Methods of modifying the Fc region to decrease binding affinity to FcγRIIB are also known in the art (e.g., U.S. Patent Publication No. 20010036459 and PCT Publication No. WO 01/79299, both to Ravetch et al., the disclosures of which are incorporated herein in their entireties). Modified antibodies having variant Fc regions with enhanced binding affinity for FcγRIIIA and/or FcγRIIA as compared with a wildtype Fc region have also been described (e.g., PCT Publication Nos. WO 2004/063351, to Stavenhagen et al., the disclosure of which is incorporated herein in its entirety).

In vitro assays known in the art can be used to determine whether anti-CD19 antibodies used in compositions and methods of the disclosure are capable of mediating ADCC. Exemplary assays are described in U.S. Pat. No. 5,500,362 or U.S. Pat. No. 5,821,337. Notably, useful effector cells for such assays include peripheral blood mononuclear cells (PBMC) and Natural Killer (NK) cells. Alternatively, or additionally, ADCC activity of the molecules of interest may be assessed in vivo. e.g., in an animal model such as that disclosed in Clynes et al. (Proc. Natl. Acad. Sci. (USA), 95:652-656 (1998)). The assay may also be performed using a commercially available kit, e.g. CytoTox 96™ (Promega).

### Exemplary Anti-CD19 Antibodies

In certain embodiments, the methods and compositions described herein utilize the anti-CD19 antibody 16C4 (see e.g., U.S. Publication No. 2008/0138336), or antigen binding fragment thereof. 16C4 is a CD19 mAb that has been shown to have potent ADCC effector function. 16C4 is the afucosylated form of the CD19 mAb anti-CD19-2, which was developed by humanization and affinity optimization of the HB12b mAb (Kansas G S and Tedder T F. J Immunol, 1991; 147:4094-4102; Yazawa et al., Proc Natl Acad Sci, 2005: 102(42):15178-15183; Herbst et al., J Pharmacol Exp Ther, 2010. 335(1):213-222).

In certain embodiments, an anti-CD19 antibody of the disclosure comprises a heavy chain comprising a CDR1 comprising the amino acid sequence of SEQ ID NO:2, a CDR2 comprising the amino acid sequence of SEQ ID NO:3, and a CDR3 comprising the amino acid sequence of SEQ ID NO:4. In other embodiments, an anti-CD 9 antibody of the disclosure comprises a heavy chain comprising a CDR1 comprising an amino acid sequence having at least 75%, 80%, 85%, 90%, 95%, 97%, 98%, or 99% identity with SEQ ID NO:2, a CDR2 comprising an amino acid sequence having at least 75%, 80%, 85%, 90%, 95%, 97%, 98%, or 99% identity with SEQ ID NO:3, and a CDR3 comprising an amino acid sequence having at least 75%, 80%, 85%, 90%, 95%, 97%, 98%, or 99% identity with SEQ ID NO:4. In certain embodiments, an anti-CD19 antibody of the disclosure comprises a heavy chain comprising a variable region comprising the amino acid sequence of SEQ ID NO:1. In other embodiments, an anti-CD19 antibody comprises a heavy chain comprising a variable region comprising an amino acid sequence having at least 75%, 80%, 85%, 90%, 95%, 97%, 98%, or 99% identity with SEQ ID NO:1.

In certain embodiments, an anti-CD19 antibody of the disclosure comprises a light chain comprising a CDR1 comprising the amino acid sequence of SEQ ID NO:6, a CDR2 comprising the amino acid sequence of SEQ ID NO:7, and a CDR3 comprising the amino acid sequence of SEQ ID NO:8. In other embodiments, an anti-CD19 antibody of the disclosure comprises a light chain comprising a CDR1 comprising an amino acid sequence having at least 75%, 80%, 85%, 90%, 95%, 97%, 98%, or 99% identity with SEQ ID NO:6, a CDR2 comprising an amino acid sequence having at least 75%, 80%, 85%, 90%, 95%, 97%, 98%, or 99% identity with SEQ ID NO:7, and a CDR3 comprising an amino acid sequence having at least 75%, 80%, 85%, 90%, 95%, 97%, 98%, or 99% identity with SEQ ID NO:8. In certain embodiments, an anti-CD19 antibody of the disclosure comprises a light chain comprising a variable region comprising the amino acid sequence of SEQ ID NO:5. In other embodiments, an anti-CD19 antibody comprises a light chain comprising a variable region comprising an amino acid sequence having at least 75%, 80%, 85%, 90%, 95%, 97%, 98%, or 99% identity with SEQ ID NO:5.

In certain embodiments, an anti-CD19 antibody of the disclosure comprises a heavy chain CDR1 comprising the amino acid sequence of SEQ ID NO:2, a heavy chain CDR2 comprising the amino acid sequence of SEQ ID NO:3, a heavy chain CDR3 comprising the amino acid sequence of SEQ ID NO:4, a light chain CDR1 comprising the amino acid sequence of SEQ ID NO:6, a light chain CDR2 comprising the amino acid sequence of SEQ ID NO:7, and a light chain CDR3 comprising the amino acid sequence of SEQ ID NO:8.

In certain embodiments, an anti-CD19 antibody of the disclosure comprises a heavy chain CDR1 comprising an amino acid sequence having at least 75%, 80%, 85%, 90%, 95%, 97%, 98%, or 99% identity with SEQ ID NO:2, a heavy chain CDR2 comprising an amino acid sequence having at least 75%, 80%, 85%, 90%, 95%, 97%, 98%, or 99% identity with SEQ ID NO:3, a heavy chain CDR3 comprising an amino acid sequence having at least 75%, 80%, 85%, 90%, 95%, 97%, 98%, or 99% identity with SEQ ID NO:4, a light chain CDR1 comprising an amino acid sequence having at least 75%, 80%, 85%, 90%, 95%, 97%, 98%, or 99% identity with SEQ ID NO:6, a light chain CDR2 comprising an amino acid sequence having at least 75%, 80%, 85%, 90%, 95%, 97%, 98%, or 99% identity with SEQ ID NO:7, and a light chain CDR3 comprising an amino acid sequence having at least 75%, 80%, 85%, 90%, 95%, 97%, 98%, or 99% identity with SEQ ID NO:8.

In certain embodiments, an anti-CD19 antibody of the disclosure comprises a heavy chain comprising a variable region comprising the amino acid sequence of SEQ ID NO:1 and a light chain comprising a variable region comprising the amino acid sequence of SEQ ID NO:5. In other embodiments, an anti-CD19 antibody of the disclosure comprises a heavy chain comprising a variable region comprising an amino acid sequence having at least 75%, 80%, 85%, 90%, 95%, 97%, 98%, or 99% identity with SEQ ID NO:1 and a light chain comprising a variable region comprising an amino acid sequence having at least 75%, 80%, 85%, 90%, 95%, 97%, 98%, or 99% identity with SEQ ID NO:5.

The present disclosure encompasses antibodies that are derivatives of antibody 16C4 that bind to human CD19. Standard techniques known to those of skill in the art can be used to introduce mutations (e.g., additions, deletions, and/or substitutions) in the nucleotide sequence encoding an antibody, including, for example, site-directed mutagenesis and PCR-mediated mutagenesis that are routinely used to generate amino acid substitutions. In one embodiment, the VH and/or VK CDRs derivatives may include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, less than 2 amino acid substitutions, or 1 amino acid substitution relative to the original VH and/or VK CDRs of the 16C4 anti-CD19 antibody. In another embodiment, the VH and/or VK CDRs derivatives may have conservative amino acid substitutions made at one or more predicted non-essential amino acid residues (e.g., amino acid residues which are not critical for the antibody to specifically bind to human CD19). Mutations can also be introduced randomly along all or part of the VII and/or VK CDR coding sequences, such as by saturation mutagenesis, and the resultant mutants can be screened for biological activity to identify mutants that retain activity. Following mutagenesis, the encoded antibody can be expressed and the activity of the antibody can be determined. The percent identity of two amino acid sequences can be determined by any method known to one skilled in the art, including, but not limited to, BLAST protein searches.

### C. Anti-CD20 Antibodies

The combination therapy described herein further comprises anti-CD20 antibodies. The CD20 antigen (also called human B-lymphocyte-restricted differentiation antigen, Bp35) is a hydrophobic transmembrane protein with a molecular weight of approximately 35 kD located on pre-B and mature B lymphocytes (Valentine et al. J. Biol. Chem. 264(19):11282-11287 (1989); Einfeld et al. EMBO J. 7(3):711-717 (1988); Clark and Ledbetter, Adv. Can. Res. 52:81-149 (1989); and Valentine et al. J. Biol. Chem. 264(19):11282-11287 (1989)). CD20 is expressed during early pre-B cell development and remains until plasma cell differentiation; it is not found on human stem cells, lymphoid progenitor cells or normal plasma cells. CD20 is present on both normal B cells as well as malignant B cells, for example the cells in B-cell non-Hodgkin's lymphoma (NHL), where CD20 is expressed on greater than 90% of NHL (Anderson et al. Blood 63(6):1424-1433 (1984)). CD20 is not found on hematopoietic stem cells, pro-B cells, normal plasma cells or other normal tissues (Tedder et al. J. Immunol. 135(2):973-979 (1985)). CD20 regulates an early step(s) in the activation process for cell cycle initiation and differentiation (Tedder et al., supra) and possibly functions as a calcium ion channel (Tedder et al. J. Cell. Biochem. 14D:195 (1990)).

Any suitable anti-CD20 antibody may be used in accordance with the methods and compositions described herein. Suitable anti-CD20 antibodies include, for example, known anti-CD20 antibodies, commercially available anti-CD20 antibodies, or anti-CD20 antibodies developed using methods well known in the art.

A CD20 antibody of the present disclosure may be a monoclonal human, humanized or chimeric anti-CD20 antibody. Anti-CD20 antibodies used in compositions and methods of the disclosure can be naked antibodies, immunoconjugates or fusion proteins. In certain embodiments, an anti-CD20 antibody of the disclosure may mediate human antibody-dependent cellular cytotoxicity (ADCC), complement-dependent cell-mediated cytotoxicity (CDC), and/or apoptosis in an amount sufficient to deplete circulating B cells. In exemplary embodiments, an anti-CD20 antibody of the disclosure is an anti-CD20 antibody that has been engineered to have enhanced ADCC activity relative to the parent antibody. Methods for creating antibody variants having enhanced ADCC activity are described above. In certain embodiments, an anti-CD20 antibody of the disclosure is an afucosylated antibody having enhanced ADCC activity.

In certain embodiments, an anti-CD20 antibody used in the compositions and methods of the disclosure may be a human, humanized or chimeric antibody having an IgG isotype, particularly an IgG1, IgG2, IgG3, or IgG4 human isotype or any IgG1, IgG2, IgG3, or IgG4 allele found in the human population.

Examples of antibodies which bind the CD20 antigen include: “C2B8” which is now called “Rituximab” (“RITUXAN™”); the yttrium-[90]-labeled 2B8 murine antibody designated “Y2B8” or “Ibritumomab Tiuxetan” (ZEVALIN™) (U.S. Pat. No. 5,736,137, expressly incorporated herein by reference); murine IgG2a “B1,” also called “Tositumomab,” (Beckman Coulter) optionally labeled with ¹³¹I to generate the “¹³¹I-B1” antibody (iodine I¹³¹ tositumomab, BEXXAR™) (U.S. Pat. No. 5,595,721, expressly incorporated herein by reference); murine monoclonal antibody “1F5” (Press et al. Blood 69(2):584-591 (1987) and variants thereof including “framework patched” or humanized 1F5 (WO03/002607, Leung, S.); ATCC deposit HB-96450: murine 2H7 and chimeric 2H7 antibody (U.S. Pat. No. 5,677,180, expressly incorporated herein by reference); humanized 2H7: huMax-CD20 (Genmab, Denmark); AME-133 (Applied Molecular Evolution); A20 antibody or variants thereof such as chimeric or humanized A20 antibody (cA20, hA20, respectively) (US 2003/0219433, Immunomedics); and monoclonal antibodies L27, G28-2, 93-1B3, B-CI or NU-B2 available from the International Leukocyte Typing Workshop (Valentine et al. In: Leukocyte Typing III (McMichael, Ed., p. 440. Oxford University Press (1987)). In an exemplary embodiment, the methods and compositions of the disclosure utilize rituximab, or an antigen binding fragment thereof, in combination with an anti-CD19 antibody, or fragment thereof.

Rituximab (RITUXAN™) is the antibody called “C2B8” in U.S. Pat. No. 5,736,137 issued Apr. 7, 1998 (Anderson et al.). RITUXAN™ is indicated for the treatment of patients with relapsed or refractory low-grade or follicular, CD20-positive, B-cell non-Hodgkin's lymphoma. In vitro mechanism of action studies have demonstrated that RITUXAN™ binds human complement and lyses lymphoid B-cell lines through complement-dependent cytotoxicity (CDC) (Reff et al. Blood 83(2):435-445 (1994)). Additionally, it has significant activity in assays for antibody-dependent cellular cytotoxicity (ADCC). More recently, RITUXAN™ has been shown to have anti-proliferative effects in tritiated thymidine incorporation assays and to induce apoptosis directly (Maloney et al. Blood 88(10):637a (1996)). In vivo preclinical studies have shown that RITUXAN™ depletes B cells from the peripheral blood, lymph nodes, and bone marrow of cynomolgus monkeys, presumably through complement and cell-mediated processes (Reff et al. Blood 83(2):435-445 (1994)). Rituximab was approved in the United States in November 1997 for the treatment of patients with relapsed or refractory low-grade or follicular CD20-B-cell non-Hodgkin's lymphoma (NHL) at a dose of 375 mg/m² weekly for four doses.

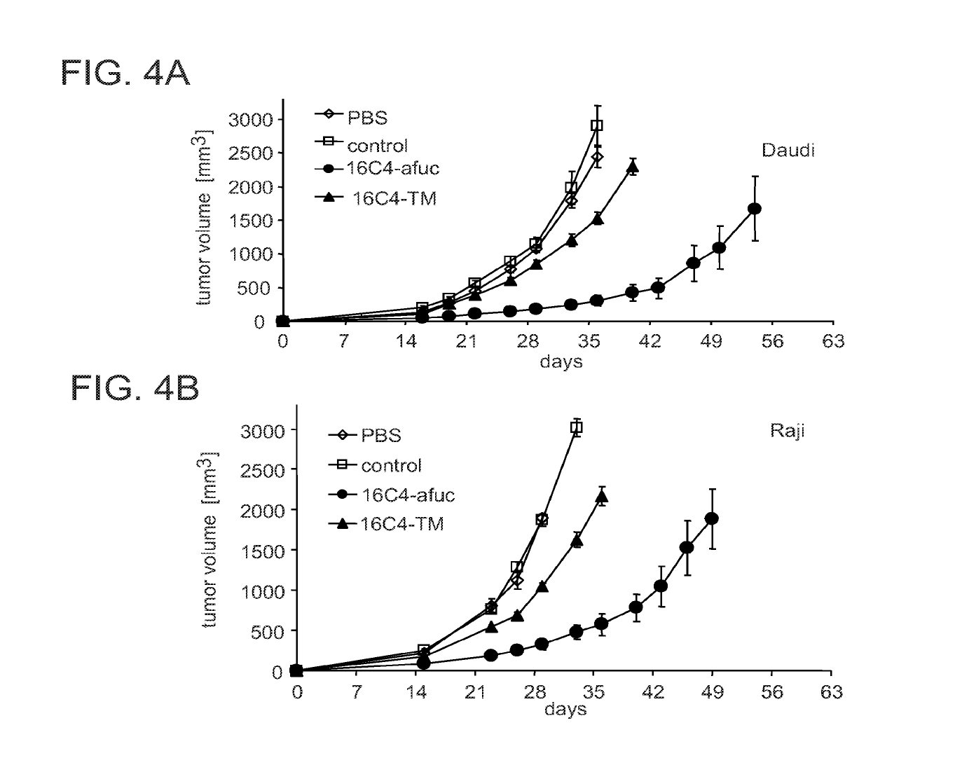

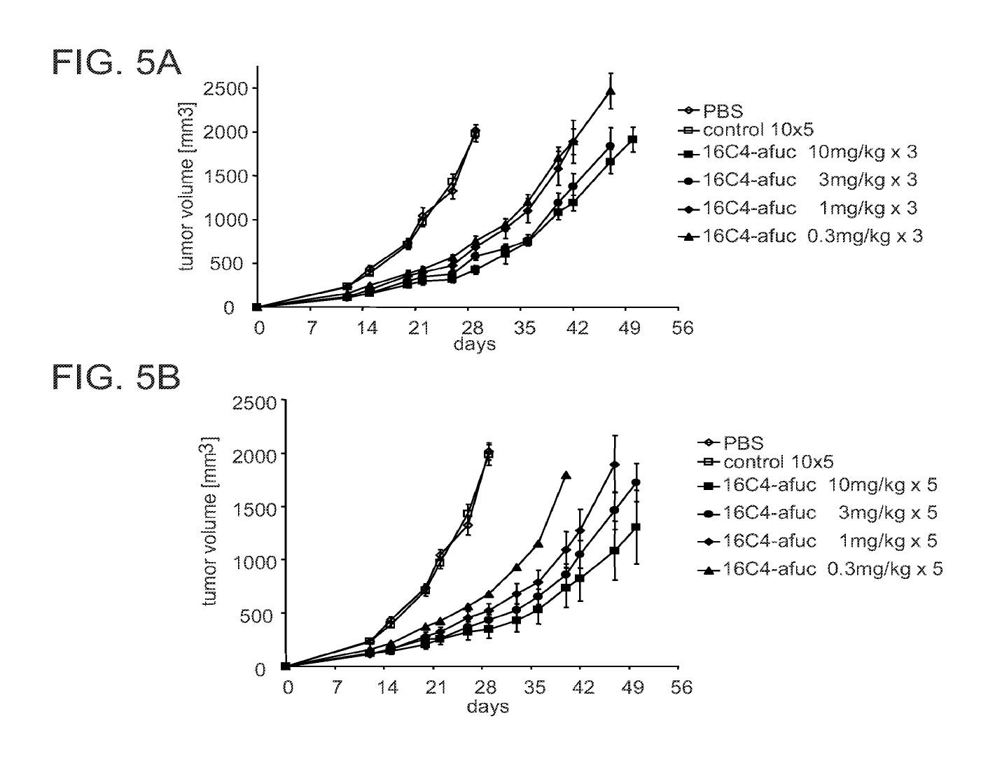

The terms “rituximab” or “RITUXAN™” herein refer to the genetically engineered chimeric murine/human monoclonal antibody directed against the CD20 antigen and designated “C2B8” in U.S. Pat. No. 5,736,137, expressly incorporated herein by reference. The complete nucleic acid and amino acid sequences for the light chain variable region and the heavy chain variable region or rituximab are disclosed in U.S. Pat. No. 5,736,137. In particular, the nucleic acid and amino acid sequences for the light chain variable region of rituximab are disclosed in FIG. 4 and SEQ ID NO:6 of U.S. Pat. No. 5,736,137. The nucleic acid and amino acid sequence for the heavy chain variable region of rituximab are disclosed in FIG. 5 and SEQ ID NO:9 of U.S. Pat. No. 5,736,137. The nucleic acid and amino acid sequences of SEQ ID NOs: 6 and 9 and FIGS. 4 and 5 of U.S. Pat. No. 5,736,137 are expressly incorporated herein by reference. Rituximab may also be made by a CHO cell transfectoma comprising the vector DNA present in the E. coli host cell deposited with the American Type Culture Collection (ATCC) under accession number. 69119. Rituximab may also be produced from hybridoma 2B8, which is deposited with the ATCC under accession number HB 11388.

### D. B Cell Malignancies

A combination therapy comprising anti-CD19 antibodies and anti-CD20 antibodies as described herein, can be used to treat B cell diseases, including B cell malignancies. The term “B cell malignancy” includes any malignancy that is derived from a cell of the B cell lineage. Exemplary B cell malignancies include, but are not limited to: B cell subtype non-Hodgkin's lymphoma (NHL) including low grade/follicular NHL, small lymphocytic (SL) NHL, intermediate grade/follicular NHL, intermediate grade diffuse NHL, high grade immunoblastic NHL, high grade lymphoblastic NHL, high grade small non-cleaved cell NHL; mantle-cell lymphoma, and bulky disease NHL; Burkitt's lymphoma; multiple myeloma; pre-B acute lymphoblastic leukemia and other malignancies that derive from early B cell precursors; common acute lymphocytic leukemia (ALL); chronic lymphocytic leukemia (CLL) including immunoglobulin-mutated CLL and immunoglobulin-unmutated CLL; hairy cell leukemia; Null-acute lymphoblastic leukemia; Waldenstrom's Macroglobulinemia; diffuse large B cell lymphoma (DLBCL) including germinal center B cell-like (GCB) DLBCL, activated B cell-like (ABC) DLBCL, and type 3 DLBCL; pro-lymphocytic leukemia; light chain disease; plasmacytoma; osteosclerotic mycloma; plasma cell leukemia; monoclonal gammopathy of undetermined significance (MGUS); smoldering multiple myeloma (SMM); indolent multiple myeloma (IMM); Hodgkin's lymphoma including classical and nodular lymphocyte pre-dominant type: lymphoplasmacytic lymphoma (LPL); and marginal-zone lymphoma including gastric mucosal-associated lymphoid tissue (MALT) lymphoma.

Treatment of relapses of these cancers is also contemplated. LPHD is a type of Hodgkin's disease that tends to relapse frequently despite radiation or chemotherapy treatment and is characterized by CD20-positive malignant cells. CLL is one of four major types of leukemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

The desired level of B cell depletion will depend on the disease. For the treatment of a B cell malignancy, it may be desirable to maximize the depletion of the B cells which are the target of the anti-CD19 and anti-CD20 antibodies of the disclosure. Thus, for the treatment of a B cell neoplasm, it is desirable that the B cell depletion be sufficient to at least prevent progression of the disease which can be assessed by the physician of skill in the art, e.g., by monitoring tumor growth (size), proliferation of the cancerous cell type, metastasis, other signs and symptoms of the particular cancer. Preferably, the B cell depletion is sufficient to prevent progression of disease for at least 2 months, more preferably 3 months, even more preferably 4 months, more preferably 5 months, even more preferably 6 or more months. In even more preferred embodiments, the B cell depletion is sufficient to increase the time in remission by at least 6 months, more preferably 9 months, more preferably one year, more preferably 2 years, more preferably 3 years, even more preferably 5 or more years. In a most preferred embodiment, the B cell depletion is sufficient to cure the disease. In preferred embodiments, the B cell depletion in a cancer patient is at least about 75% and more preferably, 80%, 85%, 90%, 95%, 99% and even 100% of the baseline level before treatment.

A patient is alleviated or successfully treated of a B cell neoplasm by the present methods of the disclosure if there is a measurable improvement in the symptoms or other applicable criteria after administration of the compositions of the disclosure compared to before treatment. The effect of treatment may be apparent within 3-10 weeks after administration of the compositions of the disclosure. The applicable criteria for each disease will be well known to the physician of skill in the appropriate art. For example, the physician can monitor the treated patient for clinical, or serologic evidence of disease such as serologic markers of disease, complete blood count including B cell count, and serum immunoglobulin levels. The patient may show observable and/or measurable reduction in or absence of one or more of the following: reduction in the number of cancer cells or absence of the cancer cells; reduction in the tumor size; inhibition (e.g., slow to some extent and preferably stop) of cancer cell infiltration into organs; inhibition (e.g., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. Preferably, after administration of the compositions of the disclosure, the improvement is at least 20% over the baseline for a particular symptom or criterion taken before treatment by the methods of the disclosure, more preferably, 25-30%, even more preferably 30-35%, most preferably 40% and above.

The parameters for assessing efficacy or success of treatment of the neoplasm will be known to the physician of skill in the appropriate disease. Generally, the physician of skill will look for reduction in the signs and symptoms of the specific disease. Parameters can include median time to disease progression, time in remission and stable disease. For B cell neoplasms, measurable criteria may include, e.g., time to disease progression, an increase in duration of overall and/or progression-free survival. In the case of leukemia, a bone marrow biopsy can be conducted to determine the degree of remission. Complete remission can be defined as the leukemia cells making up less than 5 percent of all cells found in a patient's bone marrow 30 days following treatment.

The following references describe lymphomas and CLL, their diagnoses, treatment and standard medical procedures for measuring treatment efficacy. Canellos G P, Lister, T A, Sklar J L: The Lymphomas. W.B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D: Chronic Lymphocytic Leukemia. Chap. 72, pp 1350-1362, in: Hematology, Basic Principles and Practice, 3rd ed. Hoffman et al. (editors). Churchill Livingstone, Philadelphia, 2000.

### E. Pharmaceutical Formulations

In certain aspects the invention provides pharmaceutical compositions comprising an anti-CD19 antibody, an anti-CD20 antibody, or a combination thereof and a pharmaceutically acceptable excipient. In certain embodiments, the pharmaceutical compositions of the disclosure are used as a medicament.

In certain embodiments, an anti-CD19 antibody, an anti-CD20 antibody, or a combination thereof may be formulated with a pharmaceutically acceptable carrier, excipient or stabilizer, as pharmaceutical (therapeutic) compositions, and may be administered by a variety of methods known in the art. As will be appreciated by the skilled artisan, the route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium and the like. These and additional known pharmaceutical carriers, excipients and/or additives suitable for use in the formulations described herein are known in the art, e.g., as listed in “Remington: The Science & Practice of Pharmacy”, 21^{st} ed., Lippincott Williams & Wilkins, (2005), and in the “Physician's Desk Reference”, 60 ed., Medical Economics, Montvale, N.J. (2005). Pharmaceutically acceptable carriers can be routinely selected that are suitable for the mode of administration, solubility and/or stability of the antibodies of the combination therapy, as well known those in the art or as described herein.

The formulations described herein comprise an anti-CD19 antibody, an anti-CD20 antibody, or a combination thereof in a concentration resulting in a w/v appropriate for a desired dose. In certain embodiments, an anti-CD 9 antibody or an anti-CD20 antibody is present in a formulation at a concentration of about 1 mg/ml to about 200 mg/ml, about 1 mg/ml to about 100 mg/ml, about 1 mg/ml to about 50 mg/ml, or 1 mg/ml and about 25 mg/ml. In certain embodiments, the concentration of an anti-CD19 or anti-CD20 antibody in a formulation may vary from about 0.1 to about 100 weight %. In certain embodiments, the concentration of an anti-CD19 or anti-CD20 antibody is in the range of 0.003 to 1.0 molar.

In certain embodiments, an anti-CD19 antibody and an anti-CD20 antibody are formulated together and each of the antibodies is present in a formulation at a concentration of about 1 mg/ml to about 200 mg/ml, about 1 mg/ml to about 100 mg/ml, about 1 mg/ml to about 50 mg/ml, or 1 mg/ml and about 25 mg/ml. In certain embodiments, the concentration of each of the antibodies in the formulation may vary from about 0.1 to about 100 weight %. In certain embodiments, the concentration of each of the antibodies is in the range of 0.003 to 1.0 molar.

In certain embodiments, the anti-CD19 antibody is formulated according to any of the formulations in WO 2010102276.

In one embodiment, the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). In certain specific embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

When used for in vivo administration, the formulations of the disclosure should be sterile. The formulations of the disclosure may be sterilized by various sterilization methods, including sterile filtration, radiation, etc. In one embodiment, the formulation is filter-sterilized with a presterilized 0.22-micron filter. Sterile compositions for injection can be formulated according to conventional pharmaceutical practice as described in “Remington: The Science & Practice of Pharmacy”, 21 ed., Lippincott Williams & Wilkins, (2005).

Therapeutic compositions of the present disclosure can be formulated for particular routes of administration, such as oral, nasal, pulmonary, topical (including buccal and sublingual), rectal, vaginal and/or parenteral administration. The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Formulations of the present disclosure which are suitable for topical or transdermal administration include powders, sprays, ointments, pastes, creams, lotions, gels, solutions, patches and inhalants. The antibody(ies) may be mixed under sterile conditions with a pharmaceutically acceptable carrier, and with any preservatives, buffers, or propellants which may be required (U.S. Pat. No. 7,378,110; 7,258,873; 7,135,180; US Publication No. 2004-0042972; and 2004-0042971).

The formulations may conveniently be presented in unit dosage form and may be prepared by any method known in the art of pharmacy. Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Suitable dosages may range from about 0.0001 to about 100 mg/kg of body weight or greater, for example about 0.1, 1, 10, or 50 mg/kg of body weight, with about 1 to about 10 mg/kg of body weight being preferred.

In certain embodiments, the method comprises administration of multiple doses of anti-CD20 antibody (or antigen-binding fragment thereof) in combination with multiple doses of anti-CD19 antibody (or antigen-binding fragment thereof). The method may comprise administration of 1, 2, 3, 4, 5, 6, 7, 8, 9, 10, 15, 20, 25, 30, 35, 40, or more therapeutically effective doses of a pharmaceutical composition comprising either anti-CD20 antibody (or antigen-binding fragment thereof) or anti-CD19 antibody (or antigen-binding fragment thereof), or both. The frequency and duration of administration of multiple doses of the pharmaceutical compositions can be readily determined by one of skill in the art without undue experimentation. Moreover, treatment of a subject with a therapeutically effective amount of a combination of antibodies can include a single treatment or, preferably, can include a series of treatments. For example, a subject can be treated with the combination of an anti-CD20 antibody (or antigen-binding fragment thereof) and an anti-CD19 antibody (or antigen-binding fragment thereof), where both are administered at a dose in the range of between about 1 to about 100 mg/kg body weight, once per week for between about 1 to about 10 weeks, preferably between about 2 to about 8 weeks, more preferably between about 3 to about 7 weeks, and even more preferably for about 4, 5, or 6 weeks. Treatment may occur annually to prevent relapse or upon indication of relapse.

It will also be appreciated that the effective dosage of antibodies or antigen-binding fragments thereof used for treatment may increase or decrease over the course of a particular treatment. Changes in dosage may result and become apparent from the results of diagnostic assays as described herein. Thus, in one embodiment, the dosing regimen includes administration of a therapeutically effective dose of the anti-CD20 antibody (or antigen-binding fragment thereof) in combination with a therapeutically effective dose of the anti-CD19 antibody (or antigen-binding fragment thereof), where the combination is administered on days 1, 8, 15, and 22 of a treatment period. In another embodiment, the dosing regimen includes administration of a therapeutically effective dose of the anti-CD20 antibody (or antigen-binding fragment thereof) in combination with a therapeutically effective dose of the anti-CD19 antibody (or antigen-binding fragment thereof), where the combination is administered on days 1, 2, 3, 4, 5, 6, and 7 of a week in a treatment period. Further embodiments include a dosing regimen where a therapeutically effective dose of the anti-CD20 antibody (or antigen-binding fragment thereof) is administered in combination with a therapeutically effective dose of the anti-CD19 antibody (or antigen-binding fragments thereof), where the combination is administered on days 1, 3, 5, and 7 of a week in a treatment period; a dosing regimen that includes administration of a therapeutically effective dose of the anti-CD20 antibody (or antigen-binding fragment thereof) in combination with a therapeutically effective dose of the anti-CD19 antibody (or antigen-binding fragment thereof), where the combination of antibodies is administered on days 1 and 3 of a week in a treatment period; and a preferred dosing regimen that includes administration of a therapeutically effective dose of the anti-CD20 antibody (or antigen-binding fragment thereof) in combination with the anti-CD19 antibody (or antigen-binding fragments thereof) on day 1 of any given week in a treatment period. The treatment period may comprise 1 week, 2 weeks, 3 weeks, a month, 3 months, 6 months, or a year. Treatment periods may be subsequent or separated from each other by a day, a week, 2 weeks, a month, 3 months, 6 months, or a year. Treatment using a combination of anti-CD19 antibody (or antigen-binding fragment thereof) and anti-CD20 antibody (or antigen-binding fragment thereof) may comprise administration of one or both antibodies simultaneously or concurrently, as long as the treatment includes the combination of anti-CD20 antibody (or antigen-binding fragment thereof) and anti-CD19 antibody (or antigen-binding fragment thereof) at some point during treatment. The effect of the combination therapy can also be optimized by varying the timing of administration of either the anti-CD20 antibody and/or the anti-CD19 antibody treatment. Treatment with an anti-CD20 antibody or antigen-binding fragment thereof in combination with an anti-CD19 antibody or antigen-binding fragment thereof can be simultaneous (concurrent), consecutive (sequential), or a combination thereof. Therefore, a subject undergoing combination antibody therapy can receive both the anti-CD20 antibody (or antigen-binding fragment thereof) and anti-CD19 (or antigen-binding fragment thereof) at the same time (e.g., simultaneously) or at different times (e.g., sequentially, in either order, on the same day, or on different days). Thus, in some embodiments, the anti-CD20 antibody, such as Rituximab (or antigen-binding fragment thereof) is administered simultaneously with the anti-CD19 antibody, such as the 16C4 (or antigen-binding fragment thereof). In other embodiments, the anti-CD20 antibody, such as Rituximab (or antigen-binding fragment thereof) is administered first and then the anti-CD19 antibody, such as 16C4 (or antigen-binding fragment thereof) is administered next. In yet other embodiments, the anti-CD19 antibody, such as 16C4 (or antigen-binding fragment thereof) is administered first, and the anti-CD20 antibody, such as Rituximab (or antigen-binding fragment thereof) is administered next. In some embodiments, the combination of anti-CD20 antibodies and anti-CD19 antibodies, such as Rituximab and 16C4, is given concurrently for one dosing, but other dosings include sequential administration, in either order, on the same day, or on different days. Where the anti-CD20 antibody such as Rituximab and the anti-CD19 antibody such as 16C4 are administered simultaneously, they can be administered as separate pharmaceutical compositions, each comprising either the anti-CD20 antibody (or antigen-binding fragment thereof) or the anti-CD19 antibody (or antigen-binding fragment thereof), or can be administered as a single pharmaceutical composition comprising both of these anti-cancer agents.

### EXAMPLES

The invention now being generally described, it will be more readily understood by reference to the following examples, which are included merely for purposes of illustration of certain embodiments and embodiments of the present disclosure, and are not intended to limit the invention.

### Example 1

### Anti-CD19 mAb 16C4-afuc has Potent In Vitro ADCC Activity Against Multiple B Leukemia and Lymphoma Cell Lines

16C4-afuc is the afucosylated form of mAb 16C4, which was generated by humanization and affinity maturation of the mouse IgG1 mAb HB12B. (Kansas G S and Tedder T F. J Immunol, 1991; 147:4094-4102; Yazawa et al., Proc Natl Acad Sci, 2005; 102(42):15178-15183; Herbst et al., J Pharmacol Exp Ther, 2010, 335(1):213-222). Compared to the fucosylated 16C4 mAb, 16C4-afuc has ˜9-fold increased affinity to the activating human FcγRIIIA and mouse FcγRIV and enhanced ADCC effector function. In contrast to rituximab, 16C4 does not mediate CDC. (Herbst et al., J Pharmacol Exp Ther, 2010, 335(1):213-222.)

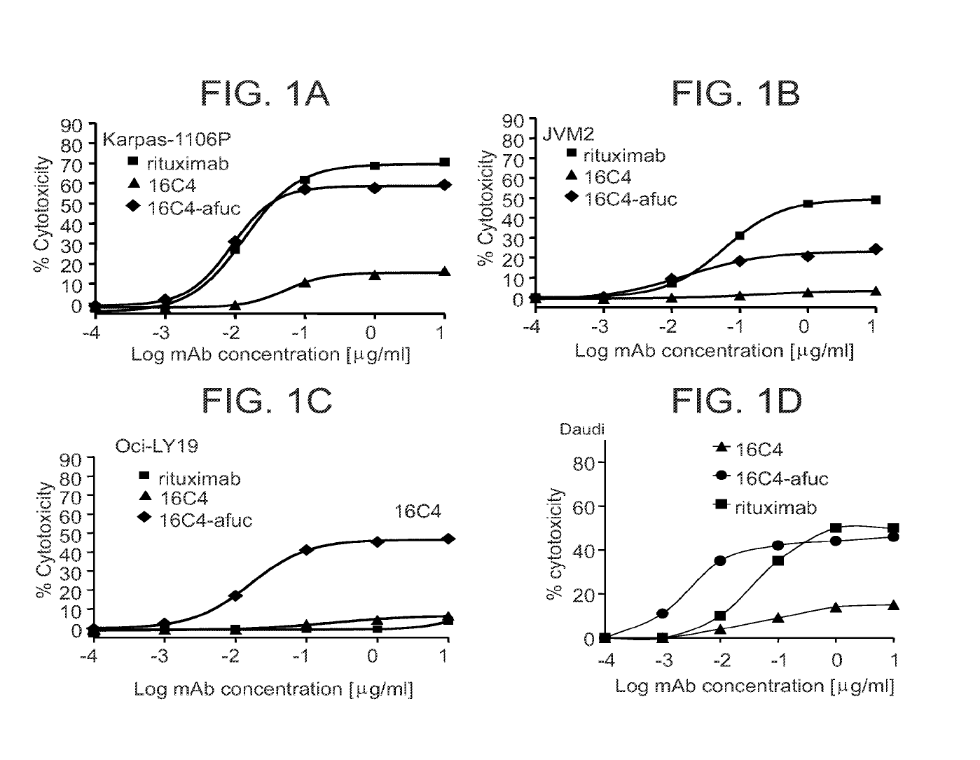

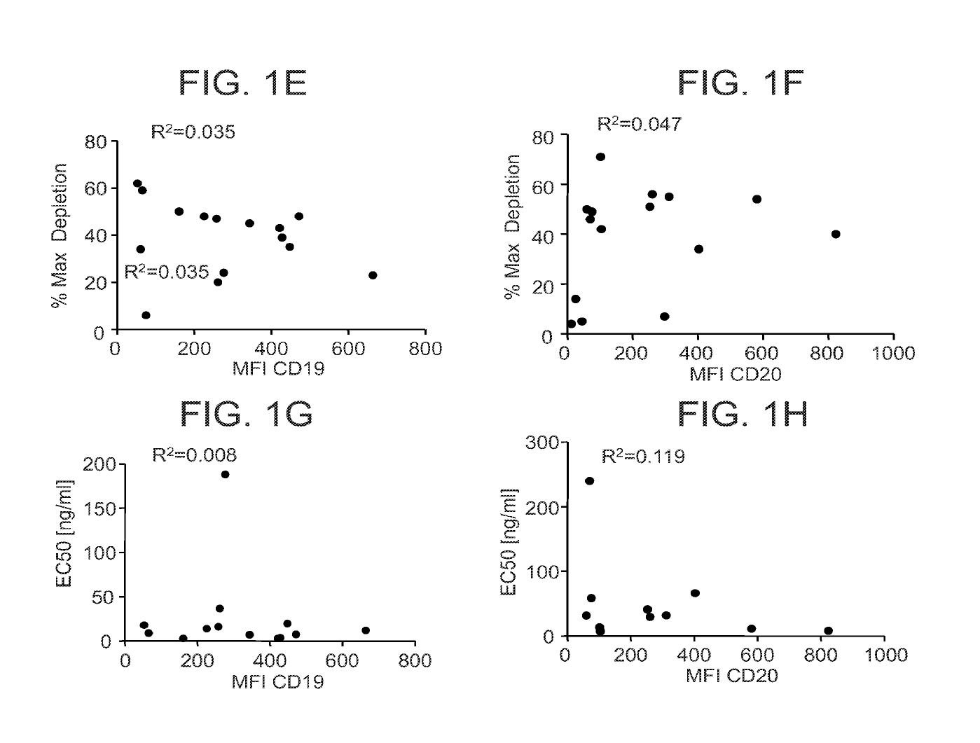

ADCC activity of 16C4-afuc was compared with that of the fucosylated precursor, mAb 16C4, in a large panel of B leukemia and lymphoma cell lines. The CD20 mAb rituximab was included in all assays as a positive control. With all cell lines tested, 16C4-afuc was significantly more potent in mediating ADCC than the parental mAb anti-CD19 mAb (FIGS. 1A-1E). When comparing the activity of 16C4-afuc with that of rituximab against the entire panel of 15 malignant B cell lines, three different activity profiles were observed: one, cell lines against which 16C4-afuc and rituximab were about equipotent, exemplified by Karpas-1106P cells (FIG. 1A), which expresses moderate to high amounts of CD19 (Table I); two, cell lines against which rituximab was more effective than 16C4-afuc such as the CLL cell line JVM2 (FIG. 1B); third, cell lines against which 16C4-afuc was more effective than rituximab, as with Oci-LY19 cells (FIG. 1C) and Daudi cells (FIG. 1D and Table 1).

The cell lines were also analyzed for their relative expression of CD19 and CD20 to determine whether the surface levels of the two antigens determines their in vitro sensitivity to CD19 and CD20 mAbs, respectively (Table 1). In FIG. 1B, the ADCC activities observed with 16C4-afuc and rituximab (maximal percentage of cytotoxicity, FIG. 1E for CD19 mAb and FIG. 1F for CD20 mAb; EC50 values, FIG. 1G for CD19 mAb and FIG. 1H for CD20 mAb) are plotted against the relative surface expression of CD19 and CD20, as determined by flow cytometry with the mAbs 16C4 and rituximab as the primary antibodies for detection. For certain cell lines the sensitivity to anti-CD19 and anti-CD20 mAbs appears to match their relative antigen expression. For example, Oci-LY19 cells have low levels of CD20 but high expression of CD19 and only respond to 16C4 treatment (Table I). The reverse was observed for Granta-519 cells, which express CD20 at high levels and are efficiently killed by rituximab, but not by 16C4-afuc. A comparison across all cell lines, however, did not show a significant correlation of antigen expression with sensitivity to ADCC mediated by either mAb. This lack of correlation is illustrated by Karpas-422 cells, which express low levels of both CD19 and CD20, and are efficiently killed by 16C4-afuc, but not by rituximab.

Together, the results demonstrate that 16C4-afuc has potent in vitro ADCC activity against multiple cell lines. With 11 of 15 cell lines tested, the ADCC activity of 16C4-afuc compared favorably to the CD20 mAb. With this diverse set of malignant B cell lines, however, there was no significant correlation of antigen expression with sensitivity to CD19 or CD20 mAb mediated ADCC.

### Example 2

### 16C4-Afuc is Effective Against Patient Derived CLL and ALL Cells In Vitro

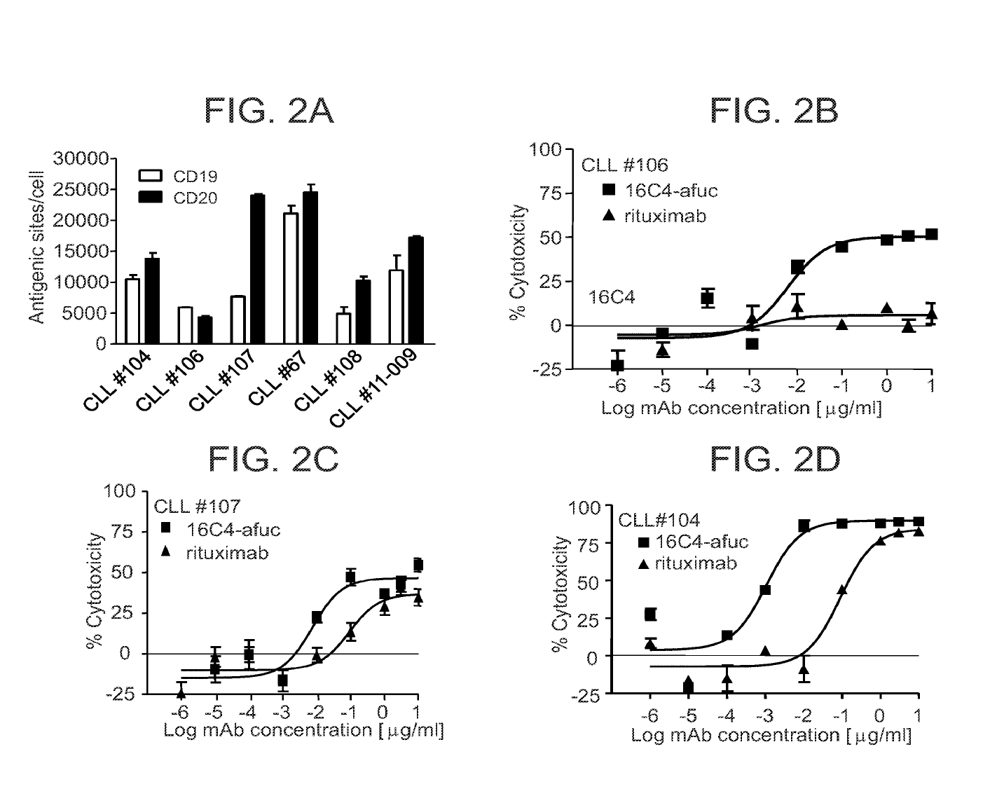

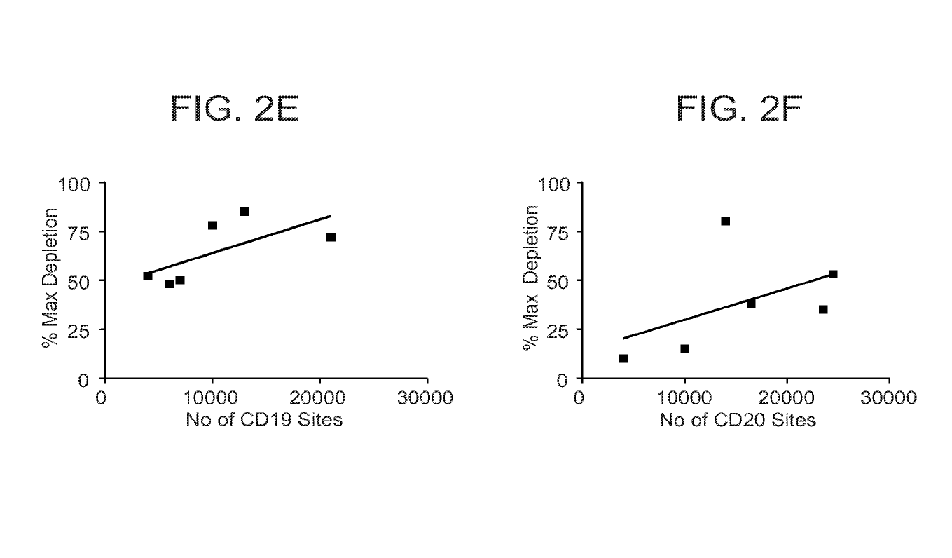

Given the activity of 16C4-afuc against B cell lines, the effects of CD19 mAb against primary leukemic cells were also examined. Six PBMC samples were obtained from patients diagnosed with CLL and the surface antigen densities for CD19 and CD20 were determined. As shown in FIG. 2A, B cells in these samples expressed CD19 and CD20 to varying degrees. In some of these samples the number of CD20 antigenic sites was greater than the number of CD19 sites. An in vitro FACS-based cytotoxicity assay was used to evaluate the ability of 16C4-afuc to kill B cells in the CLL samples, with rituximab as a positive control. FIGS. 2B-2D shows results from ADCC assays with 16C4-afuc and rituximab for three representative CLL samples (CLL #106, FIG. 2B; CLL #104, FIG. 2C; CLL #107, FIG. 2D). The EC₅₀ values for 16C4-afuc ranged from 0.007 nM to 0.063 nM. In contrast, the EC₅₀ values for rituximab ranged from 0.639 nM to 0.682 nM. The sensitivity of the CLL cells to ADCC mediated by 16C4-afuc and rituximab was compared to their surface expression of CD19 and CD20, respectively (FIGS. 2E and 2F, respectively). The results from this analysis show a clear trend towards more efficient cell killing with increasing antigen density for both CD19 and CD20. The results with these primary CLL samples also show that 16C4-afuc is more effective than rituximab in mediating depletion in vitro at relatively low levels of surface antigen expression.

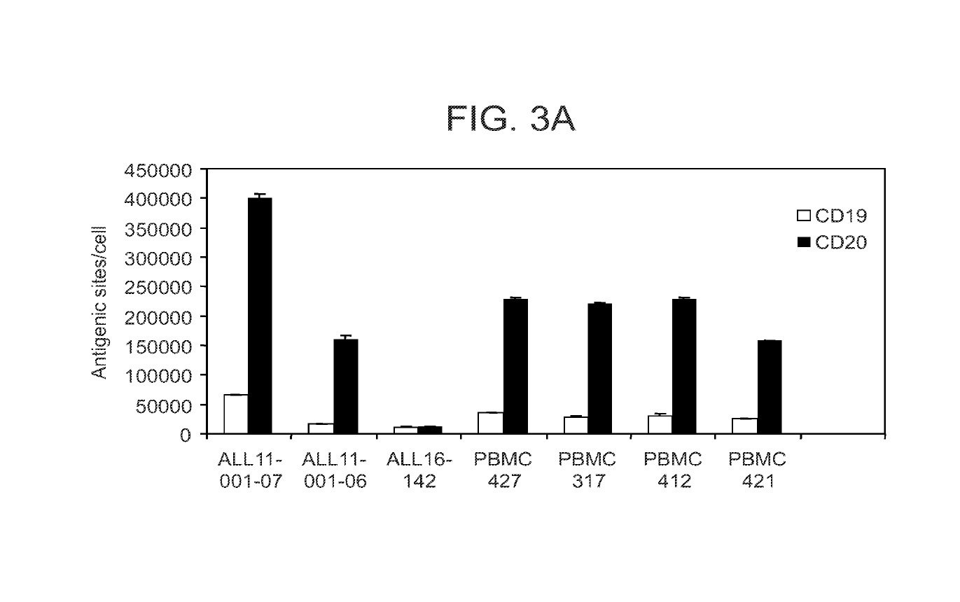

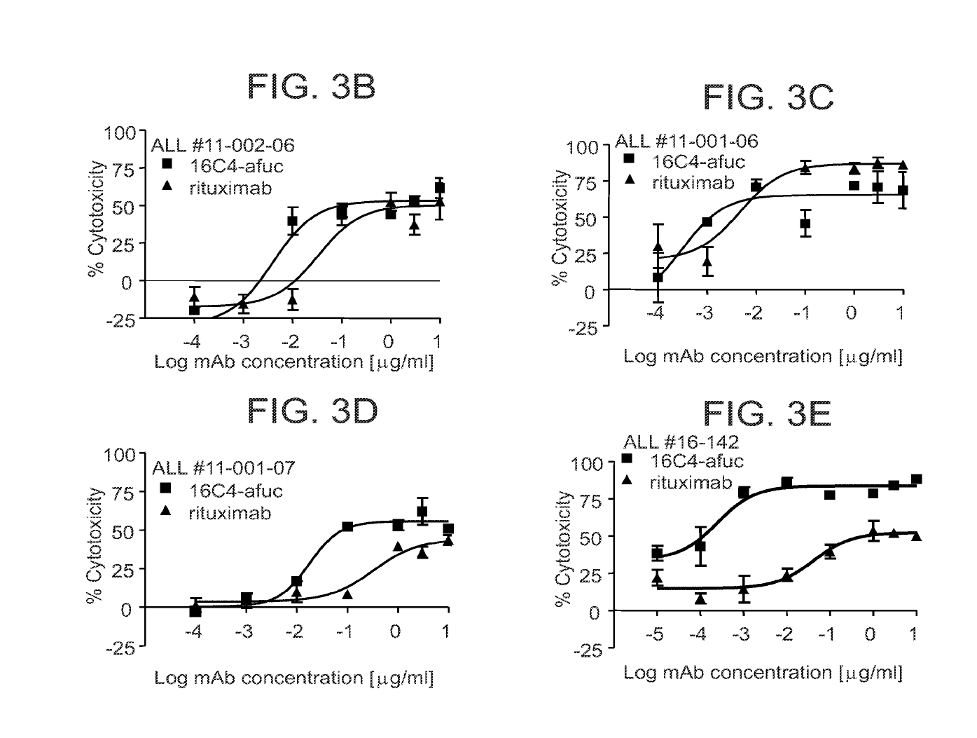

The activity of 16C4-afuc was also tested in FACS-based ADCC assays with PBMC samples from four patients with ALL. For three of these assays, there were sufficient cell numbers to determine antigen densities for CD19 and CD20, in comparison to B cells from four healthy donors (FIG. 3A). For normal peripheral blood B cells, the average density of CD19 and CD20 was determined to be ˜20,000 and ˜200,000 antigenic sites per cell, respectively. Compared to normal B cells, CD19 expression was somewhat less in two and increased by about two-fold in the third ALL sample (FIG. 3A). The number of CD20 antigenic sites, however, varied more broadly. In the ADCC B-cell depletion assays for samples from donors with ALL (FIGS. 3B-3E), the EC₅₀ values with 16C4-afuc ranged from 0.002 nM to 0.131 nM. These values were ⅙ to less than 1/100 of EC₅₀ values obtained with rituximab.

Together, the results show that 16C4-afuc effectively mediates ADCC against primary leukemic cells from patients with CLL and ALL. In these in vitro assays the CD19 mAb was more effective than the CD20 mAb rituximab. This was also the case for samples in which the level of CD20 expression was significantly higher than the levels of cell surface CD19.

Results from the preceding experiments are summarized in Table I:

| | | | rituximab | | 16C4-afuc | |
|---|---|---|---|---|---|---|
| | anti-CD 19-2 | rituximab | ADCC | % max. cell kill | ADCC | % max. cell kill |
| Cell line | MFI at 1 μg/ml | MFI at 1 μg/ml | EC50 [ng/ml] | at 10 ug/ml | EC50 [ng/ml] | at 10 ug/ml |
| Daudi (Burkitt) | 161 | 253 | 0.041 | 51 | 0.0031 | 50 |
| Granta-519 (NHL/MCL) | 75 | 403 | 0.0663 | 34 | n.d. | 6 |
| Toledo (DLCL) | 472 | 312 | 0.0319 | 55 | 0.0075 | 48 |
| Oci-LY19 (DLCL) | 258 | 12 | n.d. | 4 | 0.0163 | 47 |
| Karpas-422 (NHL) | 53 | 45 | n.d. | 5 | 0.0181 | 62 |
| Farage (DLCL) | 226 | 581 | 0.0113 | 54 | 0.0141 | 48 |
| Nalm-6 (ALL) | 448 | 26 | n.d. | 14 | 0.0199 | 35 |
| Karpas-1106P | 66 | 102 | 0.0137 | 71 | 0.009 | 59 |
| DB (DLCL) | 61 | 70 | 0.2399 | 46 | n.d. | 34 |
| RL (NHL) | 344 | 261 | 0.0296 | 56 | 0.0073 | 45 |
| Raji (Burkitt) | 422 | 823 | 0.008 | 40 | 0.003 | 43 |
| Namalwa (Burkitt) | 664 | 298 | n.d. | 7 | 0.012 | 23 |
| JVM-2 (CLL) | 277 | 76 | 0.0586 | 49 | 0.1881 | 24 |
| JVM-13 (CLL) | 262 | 60 | 0.0317 | 50 | 0.0368 | 20 |
| MeC2 (CLL) | 428 | 104 | 0.007 | 42 | 0.0039 | 39 |

### Example 3

### 16C4-Afuc Inhibits Tumor Growth in SCID-Lymphoma Models by an Fc-Dependent Mechanism

Next, the ability of 16C4-afuc to inhibit tumor growth in vivo was tested. The antitumor efficacy of 16C4 was evaluated in multiple human CD19+ lymphoma xenografts grown in SCID mice.

Some, but not all, mAbs against CD19 have anti-proliferative activity. (Ghetie et al. Blood, 1994; 83(5):1329-1336.) Previously, the mAb 16C4 was shown to inhibit proliferation of transformed B cell lines as well as primary B cell from healthy donors. (Herbst et al., J Pharmacol Exp Ther, 2010, 335(1):213-222.) In order to determine the contribution of ADCC to the anti-tumor effect, the efficacy of 16C4-afuc was compared to mAb 16C4-TM, a version of the CD19 mAb engineered for the elimination of Fc-mediated effector function. (Oganesyan et al., Acta Cryst. 2008; D64:700-704.) The antibodies were assessed in both Daudi and Raji SCID-lymphoma xenograft models at 2.5 mg/kg dosed weekly beginning on day 7 following s.c. cell implantation. Both antibodies led to a reduction in tumor growth, although the afucosylated anti-CD19 mAb was more effective than the effector-less mutant mAb 16C4-TM (FIG. 4). In the Raji model on day 33 (at the time when isotype control-treated groups had to be terminated due to tumor size), 16C4-afuc inhibited tumor growth by 84% and 16C4-TM inhibited tumor growth by 46% (FIG. 4B). By day 33 in the Daudi model, 16C4-afuc inhibited tumor growth by 88% and mAb 16C4-TM inhibited tumor growth by 39% (FIG. 4A). These results demonstrate that Fc-dependent effector function is required for effective inhibition of tumor growth in vivo. The results also show that the CD19 mAb can slow down tumor growth (albeit to a much lesser degree) in the absence of effector function, which is likely a result of the anti-proliferative activity of the mAb.

### Example 4

### 16C4-Afuc Inhibits Tumor Growth in Multiple SCID Mouse Models of Human B Cell Lymphoma

The doses of 16C4-afuc required to suppress the growth of tumors in mouse models were determined. A range of mAb doses and administration schedules were tested in the SCID/Raji s.c. xenograft model. The mAb dose range included 0.3, 1, 3, and 10 mg/kg 16C4-afuc. The dosing schedule variations included 1, 3, and 5 doses, with the first dose given on day 5 after cell implantation (FIG. 5). Overall, the in vivo efficacy of 16C4-afuc was dose and schedule dependent. In the model presented here, five doses of 16C4-afuc (FIG. 5B) resulted in stronger antitumor activity than 3 doses (FIG. 5A). However, treatment with 3 mg/kg achieved efficacy comparable to treatment with 10 mg/kg. For subsequent studies, a dose of 3 mg/kg was used, given twice per week for a total of 5 doses.

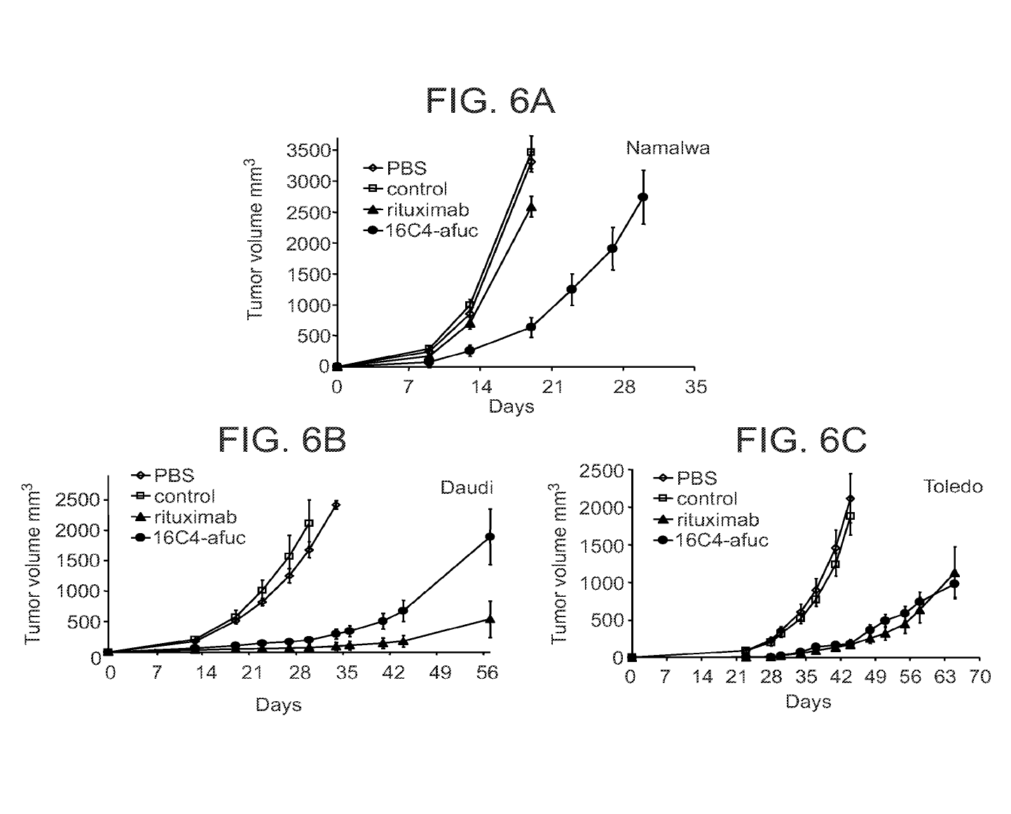

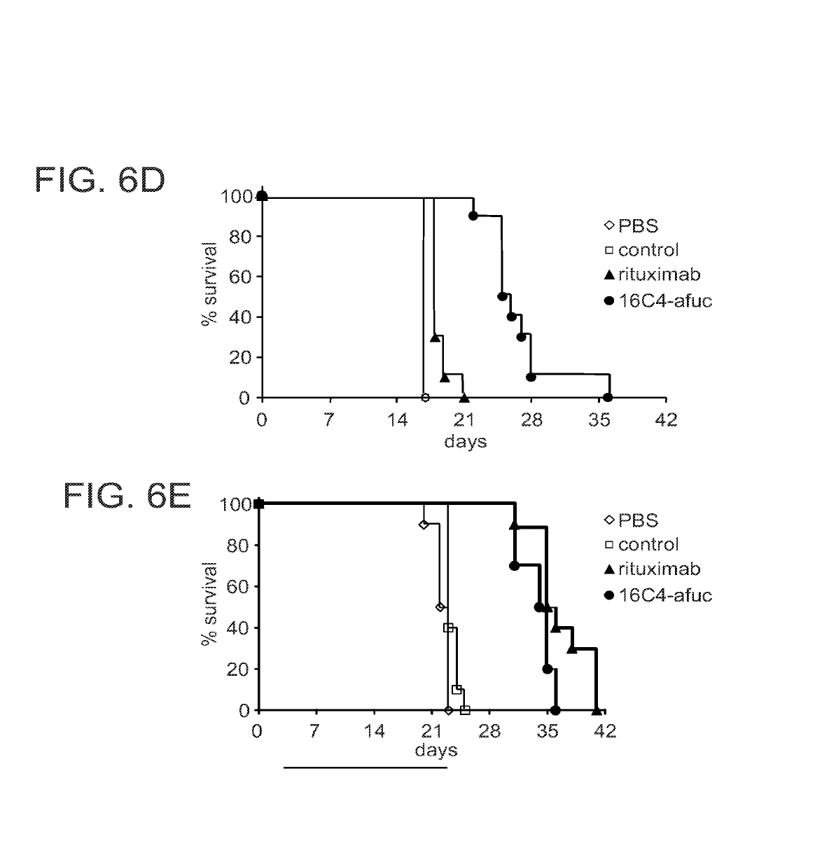

Using this dose, the efficacy of 16C4-afuc in multiple s.c. SCID lymphoma xenograft models was evaluated, with rituximab as a positive control and point of reference. FIG. 6 shows the results from tumor models with Namalwa (FIG. 6A), Daudi (FIG. 6B), and Toledo cells (FIG. 6C). In these models 16C4 inhibited tumor growth by 82%, 91%, and 88%, respectively, in comparison with results obtained for the control group. While Namalwa tumors responded poorly to rituximab treatment, Daudi xenografts were inhibited in their growth somewhat better with the CD20 mAb than with 16C4-afuc. In the Toledo tumor model the two mAbs showed comparable efficacy.

The antitumor efficacy of 16C4-afuc was further tested with Namalwa and Daudi cell xenografts in the IV disseminated tumor model with survival time or the time to paralysis as the primary endpoint. Administration of 16C4-afuc in the Namalwa (FIG. 6E) and Daudi (FIG. 6D) models increased survival by 50% and 43%, respectively, in comparison with survival observed in the control group. As with the s.c. lymphoma model, rituximab had only a minor effect in the systemic disease model with Namalwa cells. Also for the Daudi model the relative efficacy observed with mAb 16C4-afuc and rituximab recapitulated the results observed in the s.c. model. The results show that 16C4-afuc inhibits tumor growth in multiple models of B cell lymphoma, both in the s.c. and in the systemic models.

### Example 5

### Combination of 16C4-Afuc with Rituximab Results in Prolonged Suppression of Tumor Growth

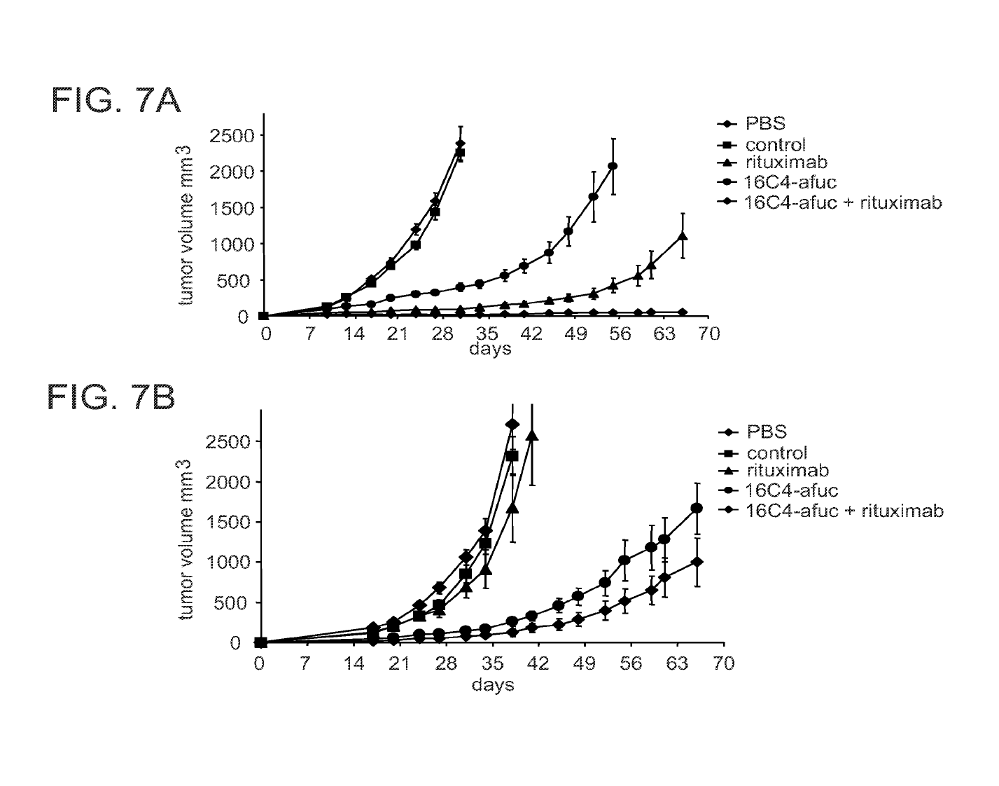

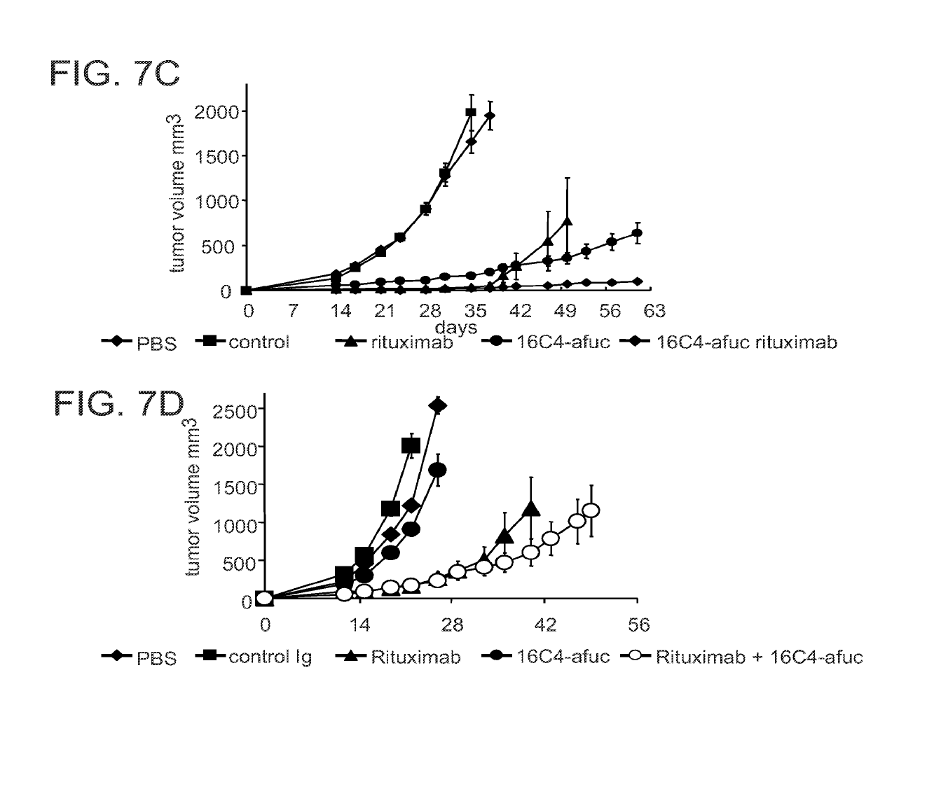

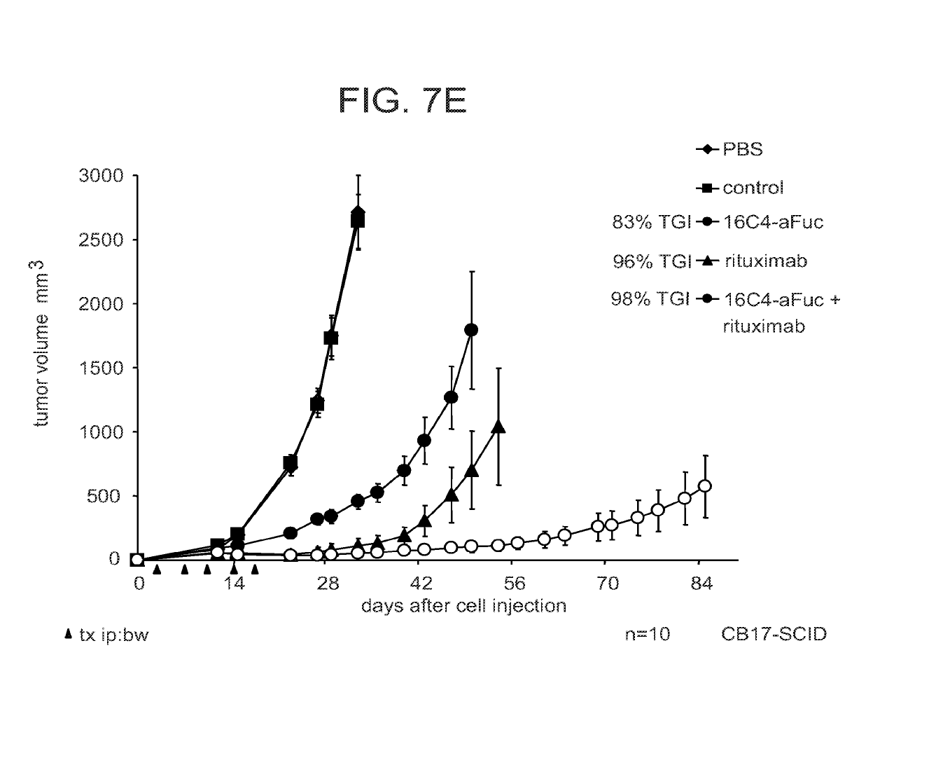

Tumor growth inhibition mediated by the combined targeting of malignant B cells with CD19 and CD20 mAbs was compared to the tumor growth inhibition resulting from targeting with either mAb alone. Four different s.c. SCID lymphoma/leukemia models (Raji, Daudi, Oci-LY19, Ramos, SUP-B15) were treated with 16C4-afuc and rituximab alone or in combination. In these experiments, rituximab was administered according to the same schedule and concentration as 16C4-afuc at 3 mg/kg for a total of 5 doses. As shown in FIG. 5, increasing the dose of 16C4-afuc from 3 mg/kg to 10 mg/kg did not result in greater inhibition of tumor growth in a SCID/Raji lymphoma model. The combination of 16C4-afuc with rituximab, however, did lead to prolonged suppression of Raji tumor growth (FIG. 7A). Similar results were obtained in the s.c. Daudi lymphoma model (FIGS. 7C), where the combination of the two mAbs resulted in greater and prolonged suppression of the s.c. lymphoma. The effect of the mAb combination was less pronounced in the Oci-LY19 s.c. xenograft model (FIG. 7B), which may have been the result of the poor activity of rituximab in this model. The CD19 mAb, however, showed good efficacy, and the combination of CD19 mAb with rituximab resulted in greater tumor suppression. Similarly, the effect of the mAb combination was less pronounced in the Ramos xenograft model (FIG. 7D), which may be been the result of the relatively poor activity of 16C4-afuc in this model. However, as seen for the Oci-LY19 model, the combination of CD19 mAb with rituximab resulted in greater tumor suppression in this model. MAb 16C4-afuc and rituximab were also tested alone and in combination in a s.c. model with SUP-B15 ALL cells (FIG. 7E). The combination of 16C4-afuc with rituximab had more pronounced tumor growth inhibition than the single agents. In addition, we tested the CD19/CD20 mAb combination treatment in a systemic Daudi tumor model. Similar to the results from the s.c. models, the combination therapy showed greater antitumor efficacy resulting in increased survival (93.5%) over single mAb-treated controls (data not shown). The results demonstrate that the combination of CD19 mAb 16C4-afuc with the CD20 mAb rituximab has greater efficacy than either mAb alone in preclinical models of human B cell lymphoma.

### Example 6

### Pharmacokinetics and Pharmacodynamics of Combination Therapy in huCD19/CD20 Double Transgenic Mice

Double transgenic animals were generated by crossing huCD19 transgenic mice with huCD20 transgenic mice. Both strains have been well characterized previously, express the transgene in a B cell restricted fashion, and have been used successfully to study B cell depletion with CD19 and CD20 mAbs, respectively. (Zhou et al., 1994, Mol Cell Biol 14:3884-3894; Ahuja et al, 2007, J Immunol 179:3351-3361; Yazawa et al, 2005, Proc Natl Acad Sci USA 102:15178-15183).

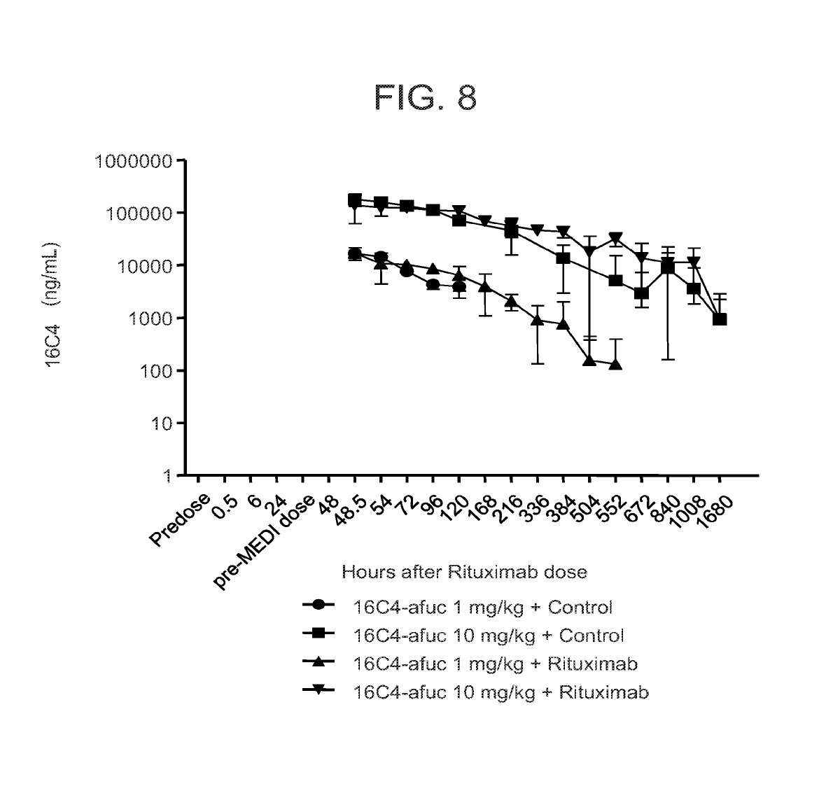

Levels of 16C4-afuc were measured in CD19/CD20 transgenic mice after a dosing regimen in which a sample of rituximab or control was administered, followed approximately 36 hours later by addition of 16C4-afuc. The levels of 16C4-afuc in the blood of the mice were measured at timepoints of up to 1680 hours. Four separate administrations were considered: 16C4-afuc (1 mg/kg)+control (e.g., no rituximab); 16C4-afuc (10 mg/kg)+control; 16C4-afuc (1 mg/kg)+rituximab (10 mg/kg); 16C4-afuc (10 mg/kg)+rituximab (10 mg/kg). FIG. 8A shows the pharmacokinetic results of the experiment. Whether administered alone or with rituximab, the higher dose of 16C4-afuc (10 mg/kg) was retained for longer in the blood than the lower dose of 16C4-afuc (1 mg/kg).

### Example 7

### B Cell Depletion in huCD19/CD20 Transgenic Mice

The extent of in vivo B cell depletion mediated by combination therapy was compared to the effects of rituximab alone (FIG. 8B). Double transgenic huCD19/CD20 mice received a dose of one of the following: (i) rituximab (10 mg/kg), (ii) 16C4-afuc (1 mg/kg); (iii) 16C4-afuc (10 mg/kg); (iv) rituximab (10 mg/kg)+16C4-afuc (1 mg/kg); (v) rituximab (10 mg/kg)+16C4-afuc (10 mg/kg). Remaining blood and spleen B cell numbers were determined by flow cytometry at intervals after the various doses were administered.

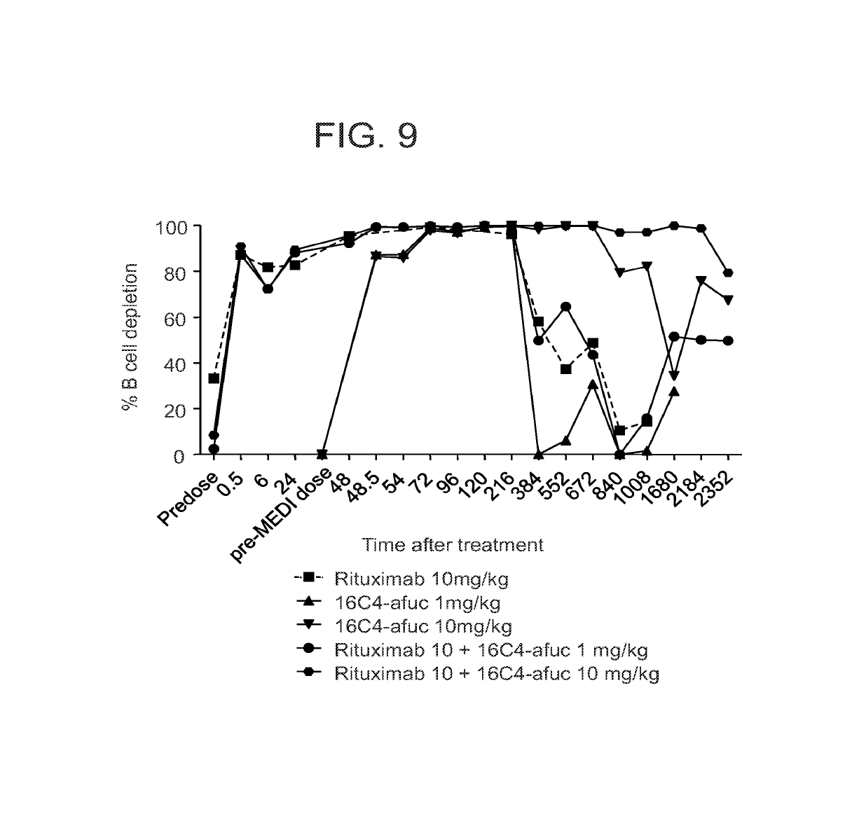

FIG. 9 shows that the highest dose of combination therapy, with rituximab (10 mg/kg)+16C4-afuc (10 mg/kg), led to the greatest percentage of B cell depletion from the blood and spleen for the longest duration. Notably, the B cell depletion drops precipitously at approximately 384 hours (or 16 days) after the dose of 16C4-afuc (1 mg/kg) has been added. A similar decline in B cell depletion is observed at approximately 840 hours (or 35 days) after rituximab (10 mg/kg) or rituximab (10 mg/kg)+16C4-afuc (1 mg/kg) has been added, although the percentage of B cell depletion appears to rise after administration of the latter dosage. When a higher dose of 16C4-afuc (10 mg/kg) has been added, the effects on B cell depletion are prolonged relative to lower doses of 16C4-afuc (whether administered singly or in combination) and relative to rituximab (10 mg/kg) alone. However, the strongest and most lasting effect is observed with the combination therapy of rituximab (10 mg/kg)+16C4-afuc (10 mg/kg).

### Material & Methods

### Cells and Reagents

The human B leukemia and lymphoma cell lines, Raji, Daudi, Ramos, Namalwa, Toledo, Farage, and RL were obtained from the American Type Culture Collection (ATCC, Manassas, Va., USA). The cell lines OCI-LY-19, Granta-519, Karpas-422, Nalm-6, Karpas-1106P, DB, JVM-2, JVM-13 and MEC2 were obtained from the Deutsche Sammlung von Mikroorganismen und Zellkulturen (DSMZ, Braunschweig, Germany). The KC1333 NK cell line (expressing human CD16) was obtained from BioWa Inc. (Princeton, N.J.). All cell lines were maintained in RPMI-1640 medium (Invitrogen, Carlsbad, Calif.) and supplemented with 10% fetal bovine serum (FBS). Blood samples were obtained from healthy donors after obtaining informed consent. The frozen ficoll-hypaque purified PBMC samples from patients diagnosed with CLL or ALL were obtained from Cureline, Inc. The humanized, affinity optimized and afucosylated CD19 mAb 16C4-afuc, mAb 16C4 (fucosylated 16C4), the human IgG1 isotype control mAb R347, and mAb 16C4-TM were provided by the MedImmune Antibody Engineering Group. The 16C4-TM mAb Fc is engineered to eliminate effector function. (Oganesyan et al., Acta Cryst, 2008: D64:700-704.) The CD20 mAb rituximab (Biogen Idec, Inc; Cambridge, Mass.) was used as positive control in in vitro and in vivo assays.

To generate a CD19 mAb with enhanced ADCC effector function, the mouse IgG1 mAb HB12b (Kansas and Tedder. J Immunol, 1991; 147:4094-4102), which recognizes human CD19, was humanized and affinity optimized, resulting in mAb 16C4. To generate a homogenously afucosylated antibody, the humanized IgG1 mAb 16C4 was expressed in a fucosyltransferase-deficient producer CHO cell line (BioWa Potelligent® Technology, BioWa Inc.; Princeton, N.J.) to generate 16C4-afuc.

### Determination of Antigen Expression on B Cell Lines

The levels of CD19 and CD20 expression on B cell lines were determined using mAb 16C4 or rituximab, respectively, as primary antibodies followed by fluorescently-labeled goat anti-human mAb. For direct binding assays, B cells were washed with PBS and resuspended in FACS buffer (PBS containing 2% FBS). The cells were incubated for 20 minutes on ice with dilutions of unlabeled mAb, washed and resuspended in PBS containing the secondary mAb. After 20 minutes on ice, the cells were washed, resuspended in FACS buffer and the fluorescence intensity on the cell surfaces was analyzed by flow cytometry. For all cell lines, maximal binding of mAb 16C4 and rituximab was achieved at concentrations of 1 μg/ml. Relative antigen expression is reported as median fluorescent intensity (MFI) in Table I.

### Determination of the Antigen Density on Normal and Malignant B Cells

The antigen densities of CD19 and CD20 on B cells from frozen PBMC from donors with CLL or ALL and PBMC samples from healthy adult donors were determined by flow cytometry using QIFIKIT® (Dako, Glostrp, Denmark) manufacturer's instructions with anti-CD19 clone HD37 and anti-CD20 clone 2H7 as the primary antibodies.

### Antibody-Dependent Cellular Cytotoxicity Assay

ADCC assays were performed with B leukemia/lymphoma cell lines as targets (T) and NK effector (E) cells at E:T ratio of 2.5:1. Cells were incubated with serial dilutions of mAb for four hours and target cell lysis was measured by detecting the release of lactate dehydrogenase (LDH) using the CytoTox 96% Non-Radioactive Cytotoxicity Assay (Promega Corp., Madison, Wis.) performed according to the manufacturer's directions. All assays were done in triplicate.

Multiparameter flow cytometry was used to quantify in vitro ADCC activity using purified PBMC from donors diagnosed with CLL or ALL. The lymphocyte content of all samples was greater than 90%. Compared to PBMC samples from healthy donors, the majority of samples from donors with CLL or ALL had low concentrations of CD56+ NK cells. Therefore, the CLL and ALL PBMC samples were supplemented with KC1333 NK cells. The frozen PBMC (CLL or ALL) samples were thawed in a 37° C. water bath, washed, and resuspended in RPMI 1640 (supplemented with 10% heat inactivated FBS and 2 mM L-glutamine), and plated at 5×10⁴ cells/well in Nunc U96-well round bottom microwell plates (ThermoFisher Scientific, Rochester, N.Y.) in a total volume of 200 μL. KC1333 NK effector cells (25×10⁴) were added to achieve an E:T ratio of 5:1. Serial dilutions of rituximab, 16C4-afuc, or of the afucosylated isotype control mAb R347 were added in 10 μl aliquots to wells (in triplicate), and the PBMC were incubated for 20 hours at 37° C. with 5% CO₂. The percentage of cytotoxicity was measured by staining cells in a cocktail of fluorescently-labeled antibodies containing anti-CD19 phycoerythrin-Cy7 (PE-Cy7), anti-CD20 Pacific Blue, anti-CD22 allophycocyanin (APC) or anti-CD22 phycoerythrin (PE), and anti-FcεR1α fluorescein isothiocyanate (FITC). As a counting standard, CountBright Absolute Counting Beads (Invitrogen. LifeTechnologies Corp., Carlsbad, Calif.) were added to determine cell concentration of cell subsets. Samples were acquired on an LSR II flow cytometer. The fluorescence activated cell sorting (FACS) data were analyzed with FlowJo (FlowJo, Ashland, Oreg.) software, version 7.2.2. The IgG1 afucosylated mAb, R347-aFuc, was used as a non-depleting treatment control and was used to define the gates. The number of absolute counting beads in each sample was quantified. The number of surviving B cells in the total CD22+ or CD20+CD22+ gates were converted to cell concentrations using the standard counting beads according to the manufacturer's instructions. B cell depletion (percentage cytotoxicity) was calculated according to the following formulae.

For rituximab:

% cytotoxity={1−[CD22+_{rituximab-treated} cells/mL]÷[CD19+CD22+_{control-treated} cells/mL]}×100.

For 16C4-afuc:

% cytotoxicity={1−[CD20+CD22+_{16C4} cells/mL]÷[CD20+CD22+_{control-treated} cells/mL]}×100.

The half maximal effective concentration (EC₅₀) of B cell cytotoxicity was calculated using a four-variable curve fit equation in GraphPad Prism v5.01 (GraphPad Software, Inc, La Jolla, Calif.).

### SCID Mouse Lymphoma Models

Inhibition of tumor growth in vivo was studied in severe combined immunodeficient (SCID)-lymphoma xenograft models. Four to six week old female CB17-SCID mice were bred at Taconic Farms (Germantown, N.Y., USA) and maintained in the Laboratory Animal Resources facility at MedImmune. All mouse experiments were conducted in accordance with IACUC approved protocols. The studies were carried out using both the localized and disseminated SCID-lymphoma xenograft models. For the localized subcutaneous (s.c.) xenograft mouse model, mice (cohorts of 10) were inoculated with 5×10⁶ tumor cells on day 0. Mice were treated with mAbs or vehicle on day 5 or 7, as indicated, by intraperitoneal (i.p.) injection. For s.c. models, tumor volume was monitored over time. For disseminated SCID mouse models, 1×10⁶ to 5×10⁶ cells were injected intravenously (i.v.) via the tail vein. At 7 days after tumor cell injection, the cohorts (10 mice each) were treated with 5 doses of mAbs at 3 mg/kg body weight, with one dose administered every 4 days. In the disseminated tumor model, survival time or the time to paralysis, a clinical symptom preceding death, was used as the endpoint.

### Pharmacokinetic/Pharmacodynamic Studies

The MSD electrochemiluminescent (ECL) immunoassay method was used to quantitate 16C4 and rituximab in mouse serum. 16C4-afuc is captured by soluble recombinant mouse anti-idiotypic antibody (D9) coated to a MA6000 MSD microtiter plate. Any bound 16C4 is then detected using biotinylated donkey anti-human IgG Fc gamma specific antibody followed by Sulfo-TAG streptavidin. This is reacted with an MSD read buffer and the plates are placed on the MSD Sector™ Imager Model 6000 reader for the generation and measurement of ECL signals. The 16C4-afuc concentration in a sample is determined by interpolation from a standard curve using a four-parameter curve fit relating the ECL counts to the concentration of 16C4.

Analysis of B Cell Numbers by Flow Cytometry huCD19/CD20 Transgenic Mice

All mouse experiments were carried out in a pathogen-free environment at the MedImmune animal facility in accordance with IACUC approved protocols. In vivo depletion of B cells by CD19 and CD20 mAbs was evaluated in huCD19/CD20 double transgenic mice, which were generated by crossing huCD19 transgenic mice with huCD20 transgenic mice (Zhou et al, 1994, Mol Cell Biol 14:3884-3894; Ahuja et al, 2007, J Immunol 179:3351-3361). Male (n=105) and female (n=105) transgenic mice (9-12 weeks old, 2 males and 2 females per time point/group) were randomized into 6 groups receiving tail vain injection of PBS, 16C4-afuc, and/or rituximab. Whole blood was collected from the orbital sinus at predetermined time points for further analysis using flow cytometry. B cell numbers were determined in each sample by staining with PerCP-Cy5.5 conjugated B220 (CD45R) and PE conjugated muCD19. B220+muCD19+ B cells were also tested for the binding of Pacific Blue conjugated huCD20 and APC-Cy7 conjugated huCD19 antibodies. Lastly, T cells were stained with FITC conjugated CD3 antibody. Samples were run on BD LSRII flow cytometer and data analyzed with FlowJo.

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

Sequences for 16C4 Antibody
SEQ ID NO: 1, VH domain
Glu Val Gln Leu Val Glu Ser Gly Gly Gly Leu Val Gln Pro Gly Gly Ser Leu Arg Leu Ser
Cys Ala Ala Ser Gly Phe Thr Phe Ser Ser Ser Trp Met Asn Trp Val Arg Gln Ala Pro Gly Lys
Gly Len Glu Trp Val Gly Arg Ile Tyr Pro Gly Asp Gly Asp Thr Asn Tyr Asn Val Lys Phe
Lys Gly Arg Phe Thr Ile Ser Arg Asp Asp Ser Lys Asn Ser Leu Tyr Leu Gln Met Asn Ser
Leu Lys The Glu Asp Thr Ala Val Tyr Tyr Cys Ala Arg Ser Gly Phe Ile Thr Thr Val Arg
Asp Phe Asp Tyr Trp Gly Gln Gly Thr Leu Val The Val Ser Ser
SEQ ID NO: 2, VH CDR1
SSWMN
SEQ ID NO: 3, VH CDR2
RIYPGDGDTNYNVKFKG
SEQ ID NO: 4, VH CDR3
SGFITTVRDFDY
SEQ ID NO: 5, VK domain
Glu Ile Val Leu Thr Gln Ser Pro Asp Phe Gln Ser Val Thr Pro Lys Glu Lys Val Thr Ile Thr
Cys Arg Ala Ser Glu Ser Val Asp Thr Phe Gly Ile Ser Phe Ile Asn Trp Phe Gln Gln Lys Pro
Asp Gln Ser Pro Lys Leu Leu Ile His Glu Ala Ser Asn Gln Gly Ser Gly Val Pro Ser Arg Phe
Ser Gly Ser Gly Ser Gly Thr Asp Phe Thr Leu Thr Ile Asn Ser Leu Glu Ala Glu Asp Ala Ala
Thr Tyr Tyr Cys Gln Gln Thr Lys Glu Val Pro Phe Thr Phe Gly Gly Gly Thr Lys Val Glu Ile
Lys
SEQ ID NO: 6, VK CDR1
RASESVDTFGISFMN
SEQ. ID NO: 7, VK CDR2
EASNQGS
SEQ ID NO: 8, VK CDR3
QQSKEVPET

1. A method for treating B cell lymphoma, comprising administering to a patient in need thereof a combination therapy comprising an anti-CD19 antibody and an anti-CD20 antibody.

2. The method of claim 1, wherein said B cell lymphoma is selected from acute lymphoblastic leukemia (ALL), chronic lymphocytic leukemia (CLL), or non-Hodgkin lymphoma (NHL).

3. The method of claim 1, wherein the combination therapy provides a synergistic therapeutic effect.

4. The method of claim 1, wherein the anti-CD19 and anti-CD20 antibodies are administered simultaneously.

5. The method of claim 1, wherein the anti-CD19 and anti-CD20 antibodies are administered sequentially.

6. The method of claim 1, wherein the anti-tumor activity is an inhibition of tumor growth.

7. The method of claim 1, wherein the anti-tumor activity is a depletion of malignant B cells.

8. The method of claim 1, wherein the combination therapy confers anti-tumor activity for at least one month longer than either said anti-CD19 antibody or said anti-CD20 antibody administered singly on a comparable dosing schedule.

9. The method of claim 1, wherein the combination therapy confers anti-tumor activity for at least six months longer than either said anti-CD19 antibody or said anti-CD20 antibody administered singly on a comparable dosing schedule.

10. The method of claim 1, wherein the combination therapy confers anti-tumor activity for at least six months.

11. The method of claim 1, wherein tumor volume in the patient increases by less than 10% during a six month period of treatment with the combination therapy.

12. The method of claim 1, wherein the anti-CD19 antibody has enhanced antibody-dependent cell-mediated cytotoxicity (ADCC) as measured in vitro.

13. The method of claim 1, wherein the anti-CD19 antibody is afucosylated.

14. The method of claim 1, wherein the anti-CD19 antibody is a human or humanized antibody.

15. The method of claim 1, wherein the anti-CD19 antibody comprises a heavy chain CDR1 comprising the amino acid sequence of SEQ ID NO: 2, a heavy chain CDR2 comprising the amino acid sequence of SEQ ID NO: 3, a heavy chain CDR3 comprising the amino acid sequence of SEQ ID NO: 4, a light chain CDR1 comprising the amino acid sequence of SEQ ID NO:6, a light chain CDR2 comprising the amino acid sequence of SEQ ID NO: 7, and a light chain CDR3 comprising the amino acid sequence of SEQ ID NO: 8.

16. The method of claim 1, wherein the anti-CD19 antibody comprises a VH domain comprising the amino acid sequence of SEQ ID NO: 1.

17. The method of claim 1, wherein the anti-CD19 antibody comprises a VL domain comprising the amino acid sequence of SEQ ID NO: 5.

18. The method of claim 1, wherein the anti-CD19 antibody comprises a VH domain comprising the amino acid sequence of SEQ ID NO: 1 and a VL domain comprising the amino acid sequence of SEQ ID NO: 5.

19. The method of claim 1, wherein the anti-CD20 antibody is rituximab.

20. A method for treating a B cell lymphoma, comprising administering to a patient in need thereof a combination therapy comprising an anti-CD19 antibody and an anti-CD20 antibody, wherein a dosage of said combination therapy has greater anti-tumor activity than a dosage of said anti-CD19 antibody that is at least two-fold higher than the dosage of the combination therapy.

21-34. (canceled)

35. A method for treating B cell lymphoma, comprising administering to a patient in need thereof a combination therapy comprising an anti-CD19 antibody and an anti-CD20 antibody, wherein said combination therapy provides anti-tumor activity for a longer duration than either said anti-CD19 antibody or said anti-CD20 antibody administered alone on a comparable dosing schedule.


SEQUENCE LISTING (8 sequences)

SEQ ID NO: 1 - protein, 121 aa, Artificial Sequence
  Description of Artificial Sequence Synthetic polypeptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 2 - protein, 5 aa, Artificial Sequence
  Description of Artificial Sequence Synthetic peptide
SSWMN

SEQ ID NO: 3 - protein, 17 aa, Artificial Sequence
  Description of Artificial Sequence Synthetic peptide
RIYPGDGDTNYNVKFKG

SEQ ID NO: 4 - protein, 12 aa, Artificial Sequence
  Description of Artificial Sequence Synthetic peptide
SGFITTVRDFDY

SEQ ID NO: 5 - protein, 111 aa, Artificial Sequence
  Description of Artificial Sequence Synthetic polypeptide
EIVLTQSPDFQSVTPKEKVTITCRASESVDTFGISFINWFQQKPDQSPKLLIHEASNQGSGVPSRFSGSGSGTDFTLTINSLEAEDAATYYCQQTKEVPFTFGGGTKVEIK

SEQ ID NO: 6 - protein, 15 aa, Artificial Sequence
  Description of Artificial Sequence Synthetic peptide
RASESVDTFGISFMN

SEQ ID NO: 7 - protein, 7 aa, Artificial Sequence
  Description of Artificial Sequence Synthetic peptide
EASNQGS

SEQ ID NO: 8 - protein, 9 aa, Artificial Sequence
  Description of Artificial Sequence Synthetic peptide
QQSKEVPFT